(19) United States
(12) Patent Application Publication
(10) Pub. No.: US 2023/0228767 A1
(43) Pub. Date: Jul. 20, 2023

(54) DETECTION OF SARS-COV-2 PROTEINS AND MITOCHONDRIAL PROTEINS IN PLASMA NEURON-DERIVED EXTRACELLULAR VESICLES AND ASTROCYTE-DERIVED EXTRACELLULAR VESICLES

(71) Applicant: Edward J. Goetzl, San Francisco, CA (US)

(72) Inventor: Edward J. Goetzl, San Francisco, CA (US)

(21) Appl. No.: 18/099,226

(22) Filed: Jan. 19, 2023

Related U.S. Application Data
(60) Provisional application No. 63/301,031, filed on Jan. 19, 2022.

(51) Int. Cl. G01N 33/68; G01N 33/569

(52) U.S. Cl. CPC G01N 33/6893; G01N 33/569

(57) ABSTRACT

The present disclosure relates to the detection of SARS-CoV-2 proteins and mitochondrial proteins in plasma neuron-derived extracellular vesicles and astrocyte-derived extracellular vesicles. The disclosure also provides compositions for detecting SARS-CoV-2 proteins and mitochondrial proteins in plasma neuron-derived extracellular vesicles and astrocyte-derived extracellular vesicles in biological samples as well as compositions and methods useful for diagnosing, prognosing, and treating long COVID-19.

### RELATED APPLICATIONS

This application claims priority to the U.S. Provisional Patent Application Ser. No. 63/301,031, filed on Jan. 19, 2022, which is hereby incorporated by reference herein in their entirety.

### FIELD OF THE INVENTION

The present disclosure relates to the detection of SARS-CoV-2 proteins and mitochondrial proteins in plasma neuron-derived extracellular vesicles and astrocyte-derived extracellular vesicles. The disclosure also provides compositions for detecting SARS-CoV-2 proteins and mitochondrial proteins in plasma neuron-derived extracellular vesicles and astrocyte-derived extracellular vesicles in biological samples as well as compositions and methods useful for diagnosing, prognosing, and treating long COVID-19.

### BACKGROUND OF THE INVENTION

A significant set of individuals infected with the Severe Acute Respiratory Syndrome coronavirus-2 (SARS-CoV-2), who develop Coronavirus Disease-2019 (COVID-19), will experience continuing or new symptoms after a four-week acute phase and often for many months (See Lopez-Leon et al. Sci Rep 2021; 11: 16144 and Michelen et al. BMJ Glob Health 2021; 6). The most disabling components of post-acute sequelae of SARS-CoV-2 (PASC) or long-COVID state are respiratory, including dyspnea, chest pain, cough and wheezing, and neurological or psychiatric, including attention deficit, memory loss, depression, confusion, anxiety, obsessive-compulsive disorders and special sensory impairments. The incidence of associated neurological or psychiatric conditions during acute COVID-19 is directly related to disease severity, but understanding the pathophysiology of such conditions in long-COVID and useful predictive biomarkers are very limited (Taquet et al. Lancet Psychiatry 2021; 8: 416-27).

In acute encephalopathic syndromes at the initial presentation of COVID-19, viral particles were detected within cytoplasmic vacuoles of brain capillary endothelial cells in the frontal lobes and RT-PCR confirmed the presence of SARS-CoV-2 in numerous regions of the brain at autopsy (Paniz-Mondolfi et al. J Med Virol 2020; 92: 699-702 and Guerrero et al. BMC Infect Dis 2021; 21: 515). The angiotensin-converting enzyme 2 (ACE2) receptor for SARS-CoV-2 also is widespread in numerous regions of the human brain, which likely promotes neurotropic distribution (Chen et al. Front Neurol 2020; 11: 573095). SARS-CoV-2 RNAs are enriched in host neural cell mitochondria and nucleoli, and both 5′- and 3′-untranslated regions contain mitochondrial localization signals (Wu et al. Nature 2020; 579: 265-9 and Wu et al. bioRxiv 2020). Numerous SARS-CoV-2 proteins interact meaningfully with host mitochondrial proteins (MPs) (Gordon et al. Nature 2020; 583: 459-68 and Gordon et al. bioRxiv 2020). For example, SARS-CoV-2 ORF9c and Nsp7 interact, respectively, with mitochondrial NDUFAF1 and NDUFAF2, which both are involved in the assembly of complex I of the mitochondrial electron transport chain. It thus appears that both SARS-CoV-2 RNA- and protein-dependent mechanisms may diminish effectiveness of neural mitochondrial pathways of defense against these viruses. Mitochondrial localization of coronaviruses and mitochondrial dependence of their replication and pathways of cellular damage have been confirmed in domestic animal infections (Lee et al. Virology 2007; 365: 419-34 and Zhang et al. Emerg Microbes Infect 2020; 9: 439-56). Coronavirus evasion of immunity also is strongly dependent on modulation of mitochondrial mechanisms (Gatti et al. Front Pharmacol 2020; 11: 578599). There have been no reliable approaches in living patients for determining the extent or mechanisms of mitochondrial involvement in the effects of central nervous system (CNS) infection by SARS-CoV-2.

Thus, there is a need in the art for biomarkers and methods for detecting SARS-CoV-2 proteins and mitochondrial proteins in plasma neuron-derived extracellular vesicles and astrocyte-derived extracellular vesicles in biological samples as well as compositions and methods useful for diagnosing and/or prognosing long COVID-19. Additionally, there is a need in the art for compositions for detecting biomarkers as well as compositions and methods useful for treating long COVID. The present disclosure meets this need by providing accurate, noninvasive methods for detecting SARS-CoV-2 proteins and mitochondrial proteins in plasma neuron-derived extracellular vesicles and astrocyte-derived extracellular vesicles in biological samples. The present disclosure further provides novel methods, assays, kits, and compositions for diagnosing, prognosing, predicting, and treating long COVID-19.

This background information is provided for the purpose of making known information believed by the applicant to be of possible relevance to the present disclosure. No admission is necessarily intended, nor should be construed, that any of the preceding information constitutes prior art against the present disclosure.

### SUMMARY OF THE INVENTION

The disclosure is based on the discovery of biomarkers from astrocyte-derived vesicles and neuron-derived vesicles that can be used to detect SARS-CoV-2 proteins and mitochondrial proteins associated with long COVID-19. These biomarkers can be used alone or in combination with one or more additional biomarkers or relevant clinical parameters in prognosis, diagnosis, or monitoring treatment of abnormalities associated with long COVID-19. The disclosure is also based on the discovery that agents that modify and/or affect the levels of SARS-CoV-2 proteins and mitochondrial proteins biomarkers from astrocyte-derived vesicles and neuron-derived vesicles are useful for predicting and/or treating long COVID-19.

Biomarkers that can be used in the practice of the disclosure include, but are not limited to, CD81, syntaphilin (SNPH), subunit 6 of NADH-ubiquinone oxidoreductase (respiratory chain complex I) (CI-6), subunit 10 of cytochrome b-cl oxidase (respiratory chain complex III) (CIII-10), Humanin, myosin VI (MYO6), mitochondrial open-reading frame of the 12S rRNA-c (MOTS-c), voltage-dependent anion-selective channel protein 1 (VDAC1), N-methyl-D-aspartate receptor 1 (NMDAR1), mitochondrial calcium uniporter (MCU), solute carrier family 8 member B1 (SLC24A6) or sodium/calcium exchanger (NCLX), translocator protein (TSOP), leucine zipper EF-hand containing transmembrane 1 protein (LETM1), SARS-CoV-2 S1 (RBD), and SARS-CoV-2 nucleocapsid (N).

In some embodiments, the disclosure provides methods comprising: a) providing a biological sample comprising vesicles from a subject; b) enriching the sample for vesicles; and c) detecting the presence of one or more biomarkers, wherein the one or more biomarker is a SARS-CoV-2 protein and/or mitochondrial protein. In other embodiments, the one or more biomarker is CD8 I, syntaphilin (SNPH), subunit 6 of NADH-ubiquinone oxidoreductase (respiratory chain complex I) (CI-6), subunit 10 of cytochrome b-cl oxidase (respiratory chain complex III) (CIII-10), Humanin, myosin VI (MYO6), mitochondrial open-reading frame of the 12S rRNA-c (MOTS-c), voltage-dependent anion-selective channel protein 1 (VDAC1), N-methyl-D-aspartate receptor 1 (NMDAR1), mitochondrial calcium uniporter (MCU), solute carrier family 8 member B1 (SLC24A6) or sodium/calcium exchanger (NCLX), translocator protein (TSOP), leucine zipper EF-hand containing transmembrane 1 protein (LETM1), SARS-CoV-2 S1 (RBD), and/or SARS-CoV-2 nucleocapsid (N). In other embodiments, the vesicles are neuron-derived extracellular vesicles (NDEVs) and/or astrocyte-derived extracellular vesicles (ADEVs). In yet other embodiments, the biological sample is selected from the list consisting of whole blood, plasma, serum, lymph, amniotic fluid, urine, and saliva. In still other embodiments, the marker is a full-size marker or a fragment of the full-size marker. In some embodiments, the detecting the presence of the marker in the biological sample comprises detecting the amount of the marker in the biological sample. In other embodiments, the subject has or is suspected of having COVID-19. In some embodiments, the subject has or is suspected of having or developing long COVID-19.

In other embodiments, the disclosure provides a method comprising: a) providing a biological sample comprising vesicles from a subject currently or previously infected with COVID-19; b) isolating vesicles from the biological sample; and c) detecting the presence of one or more biomarkers in the vesicles, wherein the one or more biomarker is a SARS-CoV-2 protein and/or mitochondrial protein. In some embodiments, the isolating vesicles from the biological sample comprises: contacting the biological sample with an agent under conditions wherein the vesicles present in the biological sample bind to the agent to form an vesicle-agent complex; and isolating the vesicles from the vesicle-agent complex to obtain a sample containing the vesicles, wherein the purity of the vesicles present in said sample are greater than the purity of the vesicles present in said biological sample. In yet other embodiments, the vesicles are neuron-derived extracellular vesicles (NDEVs) and/or astrocyte-derived extracellular vesicles (ADEVs). In still other embodiments, the one or more biomarker CD81, syntaphilin (SNPH), subunit 6 of NADH-ubiquinone oxidoreductase (respiratory chain complex I) (CI-6), subunit 10 of cytochrome b-cl oxidase (respiratory chain complex III) (CIII-10), Humanin, myosin VI (MYO6), mitochondrial open-reading frame of the 12S rRNA-c (MOTS-c), voltage-dependent anion-selective channel protein 1 (VDAC1), N-methyl-D-aspartate receptor 1 (NMDAR1), mitochondrial calcium uniporter (MCU), solute carrier family 8 member B1 (SLC24A6) or sodium/calcium exchanger (NCLX), translocator protein (TSOP), leucine zipper EF-hand containing transmembrane 1 protein (LETM1), SARS-CoV-2 S1 (RBD), and/or SARS-CoV-2 nucleocapsid (N). In other embodiments, the agent is an antibody. In some embodiments, the antibody is an anti-human CD171 (L1CAM neural adhesion protein) antibody or an anti-human glutamine aspartate transporter (GLAST) (ACSA-1) antibody. In other embodiments, the biological sample is selected from the list consisting of whole blood, plasma, serum, lymph, amniotic fluid, urine, and saliva. In some embodiments, the marker is a full-size marker or a fragment of the full-size marker. In still other embodiments, the detecting the presence of the marker in the biological sample comprises detecting: the amount of the marker in the biological sample. In some embodiments, the methods further comprise the step of determining a treatment course of action based on the detection of the one or more biomarkers. In other embodiments, the subject has or is suspected of having COVID-19. In still other embodiments, the subject has or is suspected of having or developing long COVID-19.

In other embodiments, the disclosure, provides a method for treating a subject for long COVID-19, comprising the steps of: providing a biological sample from a subject currently or previously infected with COVID-19, wherein the sample comprises vesicles; measuring the level of one or more biomarkers selected from the group consisting of SARS-CoV-2 protein and/or mitochondrial protein from the biological sample, wherein an altered level of the one or more biomarkers in the sample relative to the level in a control sample is indicative of a need for treatment; and administering an effective amount of an agent to the subject thereby treating the long COVID-19 in the subject. In some embodiments the one or more marker is CD81, syntaphilin (SNPH), subunit 6 of NADH-ubiquinone oxidoreductase (respiratory chain complex I) (CI-6), subunit 10 of cytochrome b-cl oxidase (respiratory chain complex III) (CIII-10), Humanin, myosin VI (MYO6), mitochondrial open-reading frame of the 12S rRNA-c (MOTS-c), voltage-dependent anion-selective channel protein 1 (VDAC1), N-methyl-D-aspartate receptor 1 (NMDAR1), mitochondrial calcium uniporter (MCU), solute carrier family 8 member B1 (SLC24A6) or sodium/calcium exchanger (NCLX), translocator protein (TSOP), leucine zipper EF-hand containing transmembrane 1 protein (LETM1), SARS-CoV-2 S1 (RBD), and/or SARS-CoV-2 nucleocapsid (N). In other embodiments, the vesicles are neuron-derived extracellular vesicles (NDEVs) and/or astrocyte-derived extracellular vesicles (ADEVs).

In other embodiments, the present disclosure provides a method of detecting biomarkers in a biological sample, the method comprising: a) providing; i) a biological sample comprising vesicles from a subject and ii) immunoassay reagents for detection of one or more biomarkers, wherein the one or more biomarkers is a SARS-CoV-2 protein and/or a mitochondrial protein; b) isolating vesicles from the biological sample and c) detecting the presence of one or more biomarkers selected from the group consisting of syntaphilin (SNPH), subunit 6 of NADH-ubiquinone oxidoreductase (respiratory chain complex I) (CI-6), subunit 10 of cytochrome b-cl oxidase (respiratory chain complex III) (CIII-10), Humanin, myosin VI (MYO6), mitochondrial open-reading frame of the 12S rRNA-c (MOTS-c), voltage-dependent anion-selective channel protein 1 (VDAC1), N-methyl-D-aspartate receptor 1 (NMDAR1), mitochondrial calcium uniporter (MCU), solute carrier family 8 member B1 (SLC24A6) or sodium/calcium exchanger (NCLX), translocator protein (TSOP), leucine zipper EF-hand containing transmembrane 1 protein (LETM1), SARS-CoV-2 S1 (RBD), and SARS-CoV-2 nucleocapsid (N) in the vesicles using said reagents. In other embodiments, the subject has or is suspected of having COVID-19. In some embodiments, the subject has or is suspected of having or developing long COVID-19.

In some embodiments, the reagents comprise antibodies for performing an immunoassay. In some embodiments, the immunoassay is selected from the group consisting of an ELISA, radio-immunoassay, automated immunoassay, cytometric bead assay, and immunoprecipitation assay. In other embodiments, the biological sample can be any bodily fluid comprising vesicles, including, but not limited to, whole blood, plasma, serum, lymph, amniotic fluid, urine, and saliva. In some embodiments, the marker is a full-size marker. In other embodiments said marker is a fragment of the full-size marker. In other embodiments, the detecting the presence of the marker in the biological sample comprises detecting the amount of the marker in the biological sample. In some embodiments, the method further comprises the step of determining a treatment course of action based on the detection of the marker or the diagnosis of long COVID-19.

In some embodiments, the subject has been diagnosed with COVID-19 or suspected of having COVID-19. In other embodiments, the subject is at-risk of developing long COVID-19.

In some embodiments, isolating vesicles from the biological sample comprises: contacting the biological sample with an agent under conditions wherein a vesicle present in the biological sample binds to the agent to form a vesicle-agent complex; and isolating the vesicle from the vesicle-agent complex to obtain a sample containing the vesicle, wherein the purity of the vesicles present in said sample is greater than the purity of the vesicles present in said biological sample. The agent may be an antibody that specifically binds to a vesicle surface marker (e.g., CD171 or Glutamine Aspartate Transporter (GLAST)). In some aspects of the present embodiment, the contacting comprises incubating or reacting. Example 1 describes isolation of vesicles from a biological sample, for example, by immunoabsorption using an anti-CD171 antibody and an anti-Glutamine Aspartate Transporter (GLAST) antibody specific for a vesicle surface protein.

Biomarker proteins can be measured, for example, by performing immunohistochemistry, immunocytochemistry, immunofluorescence, immunoprecipitation, Western blotting, or an enzyme-linked immunosorbent assay (ELISA). In certain embodiments, the level of a biomarker is measured with an immunoassay. For example, the level of the biomarker can be measured by contacting an antibody with the biomarker, wherein the antibody specifically binds to the biomarker, or a fragment thereof containing an antigenic determinant of the biomarker. Antibodies that can be used in the practice of the disclosure include, but are not limited to, monoclonal antibodies, polyclonal antibodies, chimeric antibodies, recombinant fragments of antibodies, Fab fragments, Fab′ fragments, F(ab′)2 fragments, Fᵥ fragments, or scFᵥ fragments. In one embodiment, the method comprises measuring amounts of an in vitro complex comprising a labeled antibody bound to an astrocyte-derived vesicle biomarker. In one aspect, the vesicle biomarker is a SARS-CoV-2 protein and/or mitochondrial protein. In other embodiments, the biomarker is CD81, syntaphilin (SNPH), subunit 6 of NADH-ubiquinone oxidoreductase (respiratory chain complex I) (CI-6), subunit 10 of cytochrome b-cl oxidase (respiratory chain complex III) (CIII-10), Humanin, myosin VI (MYO6), mitochondrial open-reading frame of the 12S rRNA-c (MOTS-c), voltage-dependent anion-selective channel protein 1 (VDAC1), N-methyl-D-aspartate receptor 1 (NMDAR1), mitochondrial calcium uniporter (MCU), solute carrier family 8 member B1 (SLC24A6) or sodium/calcium exchanger (NCLX), translocator protein (TSOP), leucine zipper EF-hand containing transmembrane 1 protein (LETM1), SARS-CoV-2 S1 (RBD), and/or SARS-CoV-2 nucleocapsid (N). In some embodiments, abnormal levels of the biomarker CD81, syntaphilin (SNPH), subunit 6 of NADH-ubiquinone oxidoreductase (respiratory chain complex I) (CI-6), subunit 10 of cytochrome b-cl oxidase (respiratory chain complex III) (CIII-10), Humanin, myosin VI (MYO6), mitochondrial open-reading frame of the 12S rRNA-c (MOTS-c), voltage-dependent anion-selective channel protein 1 (VDAC1), N-methyl-D-aspartate receptor 1 (NMDAR1), mitochondrial calcium uniporter (MCU), solute carrier family 8 member B1 (SLC24A6) or sodium/calcium exchanger (NCLX), translocator protein (TSOP), leucine zipper EF-hand containing transmembrane 1 protein (LETM1), SARS-CoV-2 S1 (RBD), and/or SARS-CoV-2 nucleocapsid (N) compared to reference value ranges of the biomarkers for a control subject indicate that the subject has long COVID-19 or is at-risk of developing long COVID-19. In some aspects, the control subject is a subject without long COVID-19. In some embodiments, decreased levels of the biomarker CD81 syntaphilin (SNPH), subunit 6 of NADH-ubiquinone oxidoreductase (respiratory chain complex I) (CI-6), subunit 10 of cytochrome b-cl oxidase (respiratory chain complex III) (CIII-10), Humanin, myosin VI (MYO6), mitochondrial open-reading frame of the 12S rRNA-c (MOTS-c), voltage-dependent anion-selective channel protein 1 (VDAC1), N-methyl-D-aspartate receptor 1 (NMDAR1), mitochondrial calcium uniporter (MCU), solute carrier family 8 member B1 (SLC24A6) or sodium/calcium exchanger (NCLX), translocator protein (TSOP), leucine zipper EF-hand containing transmembrane 1 protein (LETM1), SARS-CoV-2 S1 (RBD), and/or SARS-CoV-2 nucleocapsid (N) compared to reference value ranges of the biomarkers for a control subject indicate that the subject has long COVID-19 or is at-risk of developing long COVID-19. In some aspects, the control subject is a subject without long COVID-19.

The levels of the biomarkers from vesicles from a subject can be compared to reference value ranges for the biomarkers found in one or more samples of vesicles from one or more subjects without long COVID-19 (e.g., control sample, healthy subject without COVID-19). Alternatively, the levels of the biomarkers from vesicles from a subject can be compared to reference values ranges for the biomarkers found in one or more samples of vesicles from one or more subjects with long COVID-19.

In some embodiments, the disclosure provides a method for monitoring the efficacy of a therapy for treating long COVID-19 in a subject, the method comprising: a) providing a first biological sample comprising vesicles from the subject before the subject undergoes the therapy and a second biological sample comprising vesicles after the subject undergoes the therapy; b) isolating vesicles from the first biological sample and the second biological sample; and c) detecting one or more biomarkers, wherein the biomarker is SARS-CoV-2 protein and/or mitochondrial protein in the vesicles from the first biological sample and the second biological sample; and d) comparing the levels of the one or more biomarkers in the vesicles from the first biological sample to the levels of the one or more biomarkers for the vesicles from the second biological sample, wherein decreased levels of the one or more biomarkers in the vesicles from the second biological sample compared to the levels of the one or more biomarkers in the vesicles from the first biological sample indicate that the subject is improving, and abnormal levels of the one or more biomarkers for the vesicles from the second biological sample compared to the levels of the one or more biomarkers for the vesicles from the first biological sample indicate that the subject is worsening or not responding to the therapy. In some embodiments, the one or more biomarker is CD81, syntaphilin (SNPH), subunit 6 of NADH-ubiquinone oxidoreductase (respiratory chain complex I) (CI-6), subunit 10 of cytochrome b-cl oxidase (respiratory chain complex III) (CIII-10), Humanin, myosin VI (MYO6), mitochondrial open-reading frame of the 12S rRNA-c (MOTS-c), voltage-dependent anion-selective channel protein 1 (VDAC1), N-methyl-D-aspartate receptor 1 (NMDAR1), mitochondrial calcium uniporter (MCU), solute carrier family 8 member B1 (SLC24A6) or sodium/calcium exchanger (NCLX), translocator protein (TSOP), leucine zipper EF-hand containing transmembrane 1 protein (LETM1), SARS-CoV-2 S1 (RBD), and/or SARS-CoV-2 nucleocapsid (N). In some embodiments, the vesicles are neuron-derived extracellular vesicles (NDEVs) and/or astrocyte-derived extracellular vesicles (ADEVs).

In some embodiments, the disclosure provides a method for monitoring the efficacy of a therapy for treating long COVID-19 in a subject, the method comprising: a) providing a first biological sample comprising vesicles from the patient before the patient undergoes the therapy and a second biological sample comprising vesicles after the patient undergoes the therapy; b) isolating vesicles from the first biological sample and the second biological sample; and c) detecting one or more biomarkers selected from the group consisting of mitochondrial electron transport protein, a cytokine, and a complement protein from the vesicles from the first biological sample and the second biological sample; and d) comparing the levels of the one or more biomarkers for the vesicles from the first biological sample to the levels of the one or more biomarkers for the vesicles from the second biological sample, wherein increased levels of the one or more biomarkers for the vesicles from the second biological sample compared to the levels of the one or more biomarkers for the vesicles from the first biological sample indicate that the patient is improving, and decreased levels of the one or more biomarkers for the vesicles from the second biological sample compared to the levels of the one or more biomarkers for the vesicles from the first biological sample indicate that the patient is worsening or not responding to the therapy. In some embodiments, the one or more biomarkers are CD81, syntaphilin (SNPH), subunit 6 of NADH-ubiquinone oxidoreductase (respiratory chain complex I) (CI-6), subunit 10 of cytochrome b-cl oxidase (respiratory chain complex III) (CIII-10), Humanin, myosin VI (MYO6), mitochondrial open-reading frame of the 12S rRNA-c (MOTS-c), voltage-dependent anion-selective channel protein 1 (VDAC1), N-methyl-D-aspartate receptor 1 (NMDAR1), mitochondrial calcium uniporter (MCU), solute carrier family 8 member B1 (SLC24A6) or sodium/calcium exchanger (NCLX), translocator protein (TSOP), leucine zipper EF-hand containing transmembrane 1 protein (LETM1), SARS-CoV-2 S1 (RBD), and/or SARS-CoV-2 nucleocapsid (N). In some embodiments, the vesicles are neuron-derived extracellular vesicles (NDEVs) and/or astrocyte-derived extracellular vesicles (ADEVs).

In other embodiments, the disclosure provides a method for monitoring long COVID-19 in a subject, the method comprising: a) measuring levels of one or more biomarkers selected from the group consisting of a SARS-CoV-2 protein and/or mitochondrial protein from vesicles from a first biological sample from the subject, wherein the first biological sample is obtained from the subject at a first time point; b) measuring levels of one or more biomarkers selected from the group consisting of a SARS-CoV-2 protein and/or mitochondrial protein from vesicles from a second biological sample from the subject, wherein the second biological sample is obtained from the subject at a second (i.e., later) time point; and c) comparing the levels of the biomarkers for vesicles from the first biological sample to the levels of the biomarkers for vesicles from the second biological sample, wherein decreased levels of the one or more biomarkers selected from the group consisting of a SARS-CoV-2 protein and/or mitochondrial protein from the vesicles from the second biological sample compared to the levels of the biomarkers in the first biological sample indicate that the patient is improving, and abnormal levels of the one or more biomarkers selected from the group consisting of a SARS-CoV-2 protein and/or mitochondrial protein from the vesicles from the second biological sample compared to the levels of the biomarkers for the vesicles from the first biological sample indicate that the patient is worsening. In some embodiments, the one or more biomarkers are selected from the group consisting of CD81, syntaphilin (SNPH), subunit 6 of NADH-ubiquinone oxidoreductase (respiratory chain complex I) (CI-6), subunit 10 of cytochrome b-cl oxidase (respiratory chain complex III) (CIII-10), Humanin, myosin VI (MYO6), mitochondrial open-reading frame of the 12S rRNA-c (MOTS-c), voltage-dependent anion-selective channel protein 1 (VDAC1), N-methyl-D-aspartate receptor 1 (NMDAR1), mitochondrial calcium uniporter (MCU), solute carrier family 8 member B1 (SLC24A6) or sodium/calcium exchanger (NCLX), translocator protein (TSOP), leucine zipper EF-hand containing transmembrane 1 protein (LETM1), SARS-CoV-2 S1 (RBD), and/or SARS-CoV-2 nucleocapsid (N). In some embodiments, the vesicles are neuron-derived extracellular vesicles (NDEVs) and/or astrocyte-derived extracellular vesicles (ADEVs).

In other embodiments, the disclosure provides a method for monitoring long COVID-19 in a subject, the method comprising: a) measuring levels of one or more biomarkers selected from the group consisting of a SARS-CoV-2 protein and/or mitochondrial protein from vesicles from a first biological sample from the subject, wherein the first biological sample is obtained from the subject at a first time point; b) measuring levels of one or more biomarkers selected from the group consisting of a SARS-CoV-2 protein and/or mitochondrial protein from vesicles from a second biological sample from the subject, wherein the second biological sample is obtained from the subject at a second (i.e., later) time point; and c) comparing the levels of the biomarkers in the vesicles from the first biological sample to the levels of the biomarkers in the vesicles from the second biological sample, wherein increased levels of the one or more biomarkers selected from the group consisting of a SARS-CoV-2 protein and/or mitochondrial protein from the vesicles from the second biological sample compared to the levels of the biomarkers in the first biological sample indicate that the patient is improving, and decreased levels of the one or more biomarkers from the vesicles from the second biological sample compared to the levels of the biomarkers from the vesicles from the first biological sample indicate that the patient is worsening. In one embodiment, the vesicles are neuron-derived extracellular vesicles and/or astrocyte-derived extracellular vesicles.

In other embodiments, the disclosure provides a method for monitoring long COVID-19 in a subject, the method comprising: a) measuring levels of one or more biomarkers selected from the group consisting of a SARS-CoV-2 protein and/or mitochondrial protein from vesicles from a first biological sample from the subject, wherein the first biological sample is obtained from the subject at a first time point; b) measuring levels of one or more biomarkers selected from the group consisting of a SARS-CoV-2 protein and/or mitochondrial protein from vesicles from a second biological sample from the subject, wherein the second biological sample is obtained from the subject at a second (i.e., later) time point; and c) comparing the levels of the biomarkers in the vesicles from the first biological sample to the levels of the biomarkers in the vesicles from the second biological sample, wherein decreased levels of the one or more biomarkers selected from the group consisting of a SARS-CoV-2 protein and/or mitochondrial protein from the vesicles from the second biological sample compared to the levels of the biomarkers in the first biological sample indicate that the patient is improving, and increased levels of the one or more biomarkers from the vesicles from the second biological sample compared to the levels of the biomarkers from the vesicles from the first biological sample indicate that the patient is worsening. In one embodiment, the vesicles are neuron-derived extracellular vesicles and/or astrocyte-derived extracellular vesicles.

In yet other embodiments, the disclosure provides a method of treating a patient suspected of having long COVID-19, the method comprising: a) detecting vesicle biomarker levels in the patient or receiving information regarding the vesicle biomarker levels of the patient, as determined according to a method described herein; and b) administering a therapeutically effective amount of at least one agent that alters vesicle biomarker levels in the subject. After treatment, the method may further comprise monitoring the response of the patient to treatment. In other embodiments, the vesicles are astrocyte-derived extracellular vesicles and/or neuron-derived extracellular vesicles. In some embodiments, the vesicle biomarker is a SARS-CoV-2 protein and/or mitochondrial protein.

In other embodiments, the disclosure provides a method comprising: providing a biological sample from a subject suspected of having long COVID-19; detecting the presence or level of at least one or more biomarkers selected from the group consisting of a SARS-CoV-2 protein and/or mitochondrial protein; and administering a treatment to the subject. In one embodiment, the method further comprises administering a therapeutically effective amount of at least one agent that treats long COVID-19 to the subject if abnormal levels of the one or more biomarkers are detected in the subject. In one embodiment, the method further comprises administering a therapeutically effective amount of at least one agent that treats long COVID-19 to the subject if decreased levels of the one or more biomarkers are detected in the subject. After treatment, the method may further comprise monitoring the response of the subject to treatment. In some embodiments, the one or more biomarkers comprises CD8 I, syntaphilin (SNPH), subunit 6 of NADH-ubiquinone oxidoreductase (respiratory chain complex I) (CI-6), subunit 10 of cytochrome b-cl oxidase (respiratory chain complex III) (CIII-10), Humanin, myosin VI (MYO6), mitochondrial open-reading frame of the 12S rRNA-c (MOTS-c), voltage-dependent anion-selective channel protein 1 (VDAC1), N-methyl-D-aspartate receptor 1 (NMDAR1), mitochondrial calcium uniporter (MCU), solute carrier family 8 member B1 (SLC24A6) or sodium/calcium exchanger (NCLX), translocator protein (TSOP), leucine zipper EF-hand containing transmembrane 1 protein (LETM1), SARS-CoV-2 S1 (RBD), and/or SARS-CoV-2 nucleocapsid (N). In one embodiment, the vesicles are neuron-derived extracellular vesicles and/or astrocyte-derived extracellular vesicles.

In other embodiments, the present disclosure provides a method of treating a subject with long COVID-19, comprising: providing a biological sample from the subject; determining the level of at least one or more biomarkers selected from the list consisting of a SARS-CoV-2 protein and/or mitochondrial protein using at least one reagent that specifically binds to said biomarkers; and prescribing a treatment regimen based on the level of the one or more biomarkers. In some embodiments, the method further comprises isolating vesicles from the biological sample. In some embodiments, the biomarker CD81, syntaphilin (SNPH), subunit 6 of NADH-ubiquinone oxidoreductase (respiratory chain complex I) (CI-6), subunit 10 of cytochrome b-cl oxidase (respiratory chain complex III) (CIII-10), Humanin, myosin VI (MYO6), mitochondrial open-reading frame of the 12S rRNA-c (MOTS-c), voltage-dependent anion-selective channel protein 1 (VDAC1), N-methyl-D-aspartate receptor 1 (NMDAR1), mitochondrial calcium uniporter (MCU), solute carrier family 8 member B1 (SLC24A6) or sodium/calcium exchanger (NCLX), translocator protein (TSOP), leucine zipper EF-hand containing transmembrane 1 protein (LETM1), SARS-CoV-2 S1 (RBD), and/or SARS-CoV-2 nucleocapsid (N). In other embodiments, the vesicles are neuron-derived extracellular vesicles and/or astrocyte-derived extracellular vesicles.

In some embodiments, the disclosure provides a set of biomarkers for assessing risk of developing long COVID-19 in a subject, the set comprising one or more biomarkers selected from the group consisting of a SARS-CoV-2 protein and/or mitochondrial protein, wherein vesicle levels of the biomarkers in the set are assayed; and wherein the biomarker levels of the set of biomarkers determine the risk of developing long COVID-19 in the subject with at least 40%, 50%, 60%, 70%, 75%, 80%, 85%, 90%, 95%, 99%, or 100% specificity. In some aspects, the set of biomarkers determine the risk of developing long COVID-19 in the subject with at least 40%, 50%, 60%, 70%, 75%, 80%, 85%, 90%, 95%, 99%, or 100% sensitivity. In yet other aspects, the set of biomarkers determine the risk of developing long COVID-19 in the subject with at least 40%, 50%, 60%, 70%, 75%, 80%, 85%, 90%, 95%, 99%, or 100% accuracy. In some embodiments the biomarker comprises CD81, syntaphilin (SNPH), subunit 6 of NADH-ubiquinone oxidoreductase (respiratory chain complex I) (CI-6), subunit 10 of cytochrome b-cl oxidase (respiratory chain complex III) (CIII-10), Humanin, myosin VI (MYO6), mitochondrial open-reading frame of the 12S rRNA-c (MOTS-c), voltage-dependent anion-selective channel protein 1 (VDAC1), N-methyl-D-aspartate receptor 1 (NMDAR1), mitochondrial calcium uniporter (MCU), solute carrier family 8 member B1 (SLC24A6) or sodium/calcium exchanger (NCLX), translocator protein (TSOP), leucine zipper EF-hand containing transmembrane 1 protein (LETM1), SARS-CoV-2 S1 (RBD), and/or SARS-CoV-2 nucleocapsid (N).

In other embodiments, the disclosure provides a composition comprising at least one in vitro complex comprising a labeled antibody bound to a biomarker protein selected from the group consisting of a SARS-CoV-2 protein and/or mitochondrial protein, wherein said biomarker protein is extracted from vesicles of a subject who has been diagnosed with long COVID-19, suspected of having long COVID-19, or at risk of developing long COVID-19. The antibody may be detectably labeled with any type of label, including, but not limited to, a fluorescent label, an enzyme label, a chemiluminescent label, or an isotopic label. In some embodiments, the composition is in a detection device (i.e., device capable of detecting labeled antibody). In some embodiments, the one or more biomarkers comprise CD8I, syntaphilin (SNPH), subunit 6 of NADH-ubiquinone oxidoreductase (respiratory chain complex I) (CI-6), subunit 10 of cytochrome b-cl oxidase (respiratory chain complex III) (CIII-10), Humanin, myosin VI (MYO6), mitochondrial open-reading frame of the 12S rRNA-c (MOTS-c), voltage-dependent anion-selective channel protein 1 (VDAC1), N-methyl-D-aspartate receptor 1 (NMDAR1), mitochondrial calcium uniporter (MCU), solute carrier family 8 member B1 (SLC24A6) or sodium/calcium exchanger (NCLX), translocator protein (TSOP), leucine zipper EF-hand containing transmembrane 1 protein (LETM1), SARS-CoV-2 S1 (RBD), and/or SARS-CoV-2 nucleocapsid (N). In other embodiments, the vesicles are neuron-derived extracellular vesicles and/or astrocyte-derived extracellular vesicles.

In other embodiments, the disclosure provides a kit for predicting or monitoring long COVID-19 in a subject. In some embodiments, the kit may include a container for holding a biological sample isolated from a subject who has been diagnosed or suspected of having long COVID-19 or at risk of developing long COVID-19, at least one agent that specifically detects a biomarker of the present disclosure; and printed instructions for reacting the agent with vesicles from the biological sample or a portion of the biological sample to detect the presence or amount of at least one biomarker. In other embodiments, the kit may also comprise one or more agents that specifically bind vesicles for use in isolating vesicles from a biological sample. In yet other embodiments, the kit may further comprise one or more control reference samples and reagents for performing an immunoassay. In certain embodiments, the agents may be packaged in separate containers. In some embodiments, the kit comprises agents for measuring the levels of a SARS-CoV-2 protein and/or mitochondrial protein. In yet other embodiments, the kit further comprises an antibody that binds to a vesicle surface marker (e.g., CD171 or Glutamine Aspartate Transporter (GLAST)). In other embodiments, the vesicles are neuron-derived extracellular vesicles and/or astrocyte-derived extracellular vesicles.

In other embodiments, the disclosure provides a method for treating long COVID-19, the method comprising the steps of: providing a biological sample from a subject suspected of having long COVID-19, wherein the sample comprises vesicles; measuring the level of one or more biomarkers selected from the group consisting of a SARS-CoV-2 protein and/or mitochondrial protein from the biological sample, wherein an altered level of the one or more biomarkers in the sample relative to the level in a control sample is indicative of a need for treatment; and administering an effective amount of an agent to the subject thereby treating the long COVID-19 in the subject. In some embodiments, the one or more marker is CD81, syntaphilin (SNPH), subunit 6 of NADH-ubiquinone oxidoreductase (respiratory chain complex I) (CI-6), subunit 10 of cytochrome b-cl oxidase (respiratory chain complex III) (CIII-10), Humanin, myosin VI (MYO6), mitochondrial open-reading frame of the 12S rRNA-c (MOTS-c), voltage-dependent anion-selective channel protein 1 (VDAC1), N-methyl-D-aspartate receptor 1 (NMDAR1), mitochondrial calcium uniporter (MCU), solute carrier family 8 member B1 (SLC24A6) or sodium/calcium exchanger (NCLX), translocator protein (TSOP), leucine zipper EF-hand containing transmembrane 1 protein (LETM1), SARS-CoV-2 S1 (RBD), and/or SARS-CoV-2 nucleocapsid (N). In other embodiments, the vesicles are neuron-derived extracellular vesicles and/or astrocyte-derived extracellular vesicles.

These and other embodiments of the present disclosure will readily occur to those of skill in the art in light of the disclosure herein, and all such embodiments are specifically contemplated.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

FIGS. 1A-1G set forth data showing COVID-19-associated alterations in NDEV levels of proteins involved in mitochondrial dynamics, energy generation, metabolism, and maintenance of neuronal survival. Each point represents the value for one study participant after C81 (A) normalization (B-G). The horizontal line in each column of points depicts mean value. Statistical significance of the difference in level for each group was calculated relative to Ctl values by an unpaired t test; NS, not significant; +, p<0.05; *, p<0.01; **, p<0.001. The significance (p values) of differences between participants who had PASC w/neuropsychiatric (NP) findings and those who had PASC w/o NP and between participants who had PASC w/NP and those who had PASC w/severe NP, respectively, were: <0.01 and <0.01 for CD81 (A); <0.0001 and NS for SNPH (B); NS and <0.05 for Myo6 (C); <0.05 and NS for CI-6 (D); <0.05 and <0.05 for CIII-10 (E); <0.01 and <0.001 for MOTS-c (F); and <0.01 and <0.001 for Humanin (G). NP=neuropsychiatric abnormalities.

FIGS. 2A-2F set forth data showing COVID-19-associated alterations in NDEV levels of proteins in mitochondrial ion channels and translocators. Each point represents the value for one study participant after C81 normalization. The horizontal line depicts mean value. Statistical significance of the difference in level for each group was calculated relative to Ctl values by an unpaired t test; NS, not significant; +, p<0.05; *, p<0.01; **, p<0.001. The significance (p values) of differences between participants who had PASC w/NP and those who had PASC w/o NP and between participants who had PASC w/NP and those who had PASC w/severe NP, respectively, were: NS and <0.05 for VDAC1 (A); <0.05 and <0.001 for MCU (B); <0.05 and <0.05 for NCLX (C); <0.01 and <0.01 for LETM1 (D); NS and NS for NMDAR1 (E); and NS and NS for TSOP (F). NP=neuropsychiatric abnormalities.

FIGS. 3A-3B set forth data showing COVID-19-associated alterations in levels of SARS-CoV-2 proteins 51 (receptor binding domain, RBD) (A) and nucleocapsid (N) (B) of NDEV anchored to plate wells by anti-L1CAM antibody and permeabilized prior to antibody staining for an ELISA. Each point represents the value for one study participant (Table 1). The horizontal line depicts mean value. Statistical significance of the difference in level for each group relative to Ctl value was calculated by an unpaired t test; +, p<0.05; *, p <0.01; **, p<0.001. The significance of differences between group NDEV levels of 51 RBD and Ctl levels in A are: p<0.0001 for PASC w/o NP and PASC w/NP, and p=0.0004 for PASC w/severe NP. Statistical significance of differences between group NDEV levels of N protein and Ctl levels in B are: p=0.035 for PASC w/o NP, p=0.004 for PASC w/NP, and p=0.004 for PASC w/severe NP.

FIGS. 4A-4B set forth data showing COVID-19-associated alterations in levels of SARS-CoV-2 proteins 51 (receptor binding domain, RBD) (A) and nucleocapsid (N) (B) of NDEV anchored to plate wells by anti-NCAM-1 antibody and permeabilized prior to antibody staining for an ELISA. Each point represents the value for one study participant (Table 1). The horizontal line depicts mean value. Statistical significance of the difference in level for each group relative to Ctl value was calculated by an unpaired t test; +, p<0.05; *, p <0.01; **, p<0.001. The significance of differences between group NDEV levels of 51 RBD and Ctl levels in A are: p=0.023 for PASC w/o NP, p<0.0001 for PASC w/NP, and p=0.003 for PASC w/severe NP. Statistical significance of differences between group NDEV levels of N protein and Ctl levels in B are: p=0.0003 for PASC w/o NP, p=0.004 for PASC w/NP, and p=0.005 for PASC w/severe NP.

FIGS. 5A-5B set forth data showing Detection of SARS-CoV-2 51 (RBD) and N proteins in permeabilized plasma NDEVs of COVID-19 patients (Table 1). Left-hand pairs of images are NDEVs co-stained with Abcam rabbit anti-L1CAM monoclonal antibody (magenta) and Cell Signaling Technology mouse anti-CoV-1/2 S1 protein monoclonal antibody (left of pair) or Cell Signaling Technology mouse anti-CoV-1/2 N protein monoclonal antibody (right of pair) (green). Right-hand pairs of images are NDEVs co-stained with Santa Cruz Biotechnologies mouse anti-NCAM1 monoclonal antibody (magenta) and Cell Signaling Technology mouse anti-CoV-1/2 S1 protein monoclonal antibody (left of pair) or Cell Signaling Technology mouse anti-CoV-1/2 N protein monoclonal antibody (right of pair) (green). Horizontal scale in the boxed-in areas=200 nm. Sources of NDEVs are: Controls never exposed to SARS-CoV-2, chronic stage of acute COVID-19 without any manifestation of PASC, PASC with neuropsychiatric manifestations (NP), and PASC without NP.

FIGS. 6A-6C set forth data showing ELISA-quantified levels of extracted CD81 EV marker (A) and of CD81 level normalized alterations in levels of extracted SARS-CoV-2 proteins S1 (receptor binding domain, RBD) (B) and nucleocapsid (N) (C) in NDEV isolated with anti-L1CAM antibody from acutely infected COVID-19 subjects. Each point represents the value for one study participant with acute COVID-19 or one Ctl. The horizontal line in each cluster depicts mean value. Statistical significance of the difference in level for each group relative to Ctl level was calculated by an unpaired t test; **, p<0.001. The group mean±SEM NDEV levels of S1(RBD) protein in B are: 59.5±5.29 pg/ml for Ctl, 1839±205 pg/ml for PASC, and 1913±142 pg/ml for No PASC. The group mean±SEM NDEV levels of Nucleocapsid protein in C are: 193±9.19 pg/ml for Ctl, 4881±524 pg/ml for PASC, and 5029±353 pg/ml for No PASC.

FIGS. 7A-7C set forth data showing ELISA-quantified levels of extracted CD81 EV marker (A) and of CD81-normalized alterations in levels of SARS-CoV-2 proteins S1 (receptor binding domain, RBD) (B) and nucleocapsid (N) (C) in NDEV isolated with anti-NCAM1 antibody from acutely infected COVID-19 subjects. Each point represents the value for one study participant with acute COVID-19. The horizontal line in each cluster depicts mean value. Statistical significance of the difference in level for each group relative to Ctl level was calculated by an unpaired t test; **, p<0.001. The group mean±SEM NDEV levels of S1(RBD) protein in B are: 75.3±9.28 pg/ml for Ctl, 1117±74.6 pg/ml for PASC, and 1073±62.8 pg/ml for No PASC. The group mean±SEM NDEV levels of Nucleocapsid protein in C are: 155±8.17 pg/ml for Ctl, 3390±120 pg/ml for PASC, and 3374±196 pg/ml for No PASC.

FIGS. 8A-8H set forth data showing alterations in NDEV levels of some mitochondrial proteins at the time of initial COVID-19 infection appear to predict risk for PASC. Each point represents the value for one study participant after C81 normalization. The horizontal line depicts mean value. Statistical significance of the difference in level of a mitochondrial protein for each PASC and No PASC group relative to control (C) values was calculated by an unpaired t test; +, p<0.05; *, p<0.01; **, p<0.001. The significance (p values) of differences between levels of a protein for participants who developed PASC or did not develop PASC (No PASC) and those of uninfected C participants, respectively, were: <0.0001 and 0.1267 for humanin (A); <0.0001 and 0.2029 for mitochondrial open reading frame of the 12S rRNA-c (MOTS-c) (B); <0.0001 and <0.0312 (not significant after Bonferroni correction) for voltage-dependent anion-selective channel protein 1 (VDAC1) (C); <0.0001 and <0.0001 for sterile Alpha and TIR motif-containing protein 1 (SARM1) (D); 0.0004 and 0.0046 for syntaphilin (SNPH) (E); 0.0074 and <0.0001 for N-methyl-D-aspartate receptor 1 (NMDAR1) (F); <0.0001 and <0.0001 for subunit 10 of cytochrome b-cl oxidase (respiratory chain complex III) (CIII-10) (G); and 0.862 and 0.994 for transcription factor A of mitochondria (TFAM) (H). The differences between PASC and No PASC levels were significant (after Bonferroni correction) for humanin, MOTS-c, SARM-1 and VDAC1 (p=0.0004 to <0.0001). The differences between PASC and No PASC levels were not significant (p all >0.12) for humanin, MOTS-c, SARM-1 and VDAC1.

Each of the limitations of the disclosure can encompass various embodiments of the disclosure. It is, therefore, anticipated that each of the limitations of the disclosure involving any one element or combinations of elements can be included in each aspect of the disclosure. This disclosure is not limited in its application to the details of construction and the arrangement of components set forth in the following description. The disclosure is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items. It must be noted that as used herein and in the appended claims, the singular forms “a,” “an,” and “the” include plural references unless context clearly dictates otherwise. Thus, for example, a reference to “a fragment” includes a plurality of such fragments, a reference to an “antibody” is a reference to one or more antibodies and to equivalents thereof known to those skilled in the art, and so forth.

### DESCRIPTION OF THE INVENTION

It is to be understood that the disclosure is not limited to the particular methodologies, protocols, cell lines, assays, and reagents described herein, as these may vary. It is also to be understood that the terminology used herein is intended to describe particular embodiments of the present disclosure, and is in no way intended to limit the scope of the present disclosure as set forth in the appended claims.

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present disclosure, the preferred methods, devices, and materials are now described. All publications cited herein are incorporated herein by reference in their entirety for the purpose of describing and disclosing the methodologies, reagents, and tools reported in the publications that might be used in connection with the disclosure. Nothing herein is to be construed as an admission that the disclosure is not entitled to antedate such disclosure by virtue of prior disclosure.

The practice of the present disclosure will employ, unless otherwise indicated, conventional methods of chemistry, biochemistry, molecular biology, cell biology, genetics, immunology and pharmacology, within the skill of the art. Such techniques are explained fully in the literature. See, e.g., Gennaro, A.R., ed. (1990) Remington's Pharmaceutical Sciences, 18th ed., Mack Publishing Co.; Colowick, S. et al., eds., Methods In Enzymology, Academic Press, Inc.; Handbook of Experimental Immunology, Vols. I-IV (D.M. Weir and C.C. Blackwell, eds., 1986, Blackwell Scientific Publications); Maniatis, T. et al., eds. (1989) Molecular Cloning: A Laboratory Manual, 2nd edition, Vols. I-III, Cold Spring Harbor Laboratory Press; Ausubel, F. M. et al., eds. (1999) Short Protocols in Molecular Biology, 4th edition, John Wiley & Sons; Ream et al., eds. (1998) Molecular Biology Techniques: An Intensive Laboratory Course, Academic Press); PCR (Introduction to Biotechniques Series), 2nd ed. (Newton & Graham eds., 1997, Springer Verlag).

The present disclosure relates, in part, to the discovery that vesicle biomarkers can be used to detect pathogenesis of long COVID-19. The inventor has demonstrated that diverse abnormalities in COVID-19 patients, including abnormal neuron-derived extracellular vesicle and astrocyte-derived extracellular vesicle levels of SARS-CoV-2 proteins and mitochondrial proteins (see, e.g., Example 1).

The present disclosure also provides agents for use in the methods described herein. Such agents may include small molecule compounds; peptides and proteins including antibodies or functionally active fragments thereof.

The present disclosure further provides kits for identifying a subject at risk of long COVID-19 or prescribing a therapeutic regimen or predicting benefit from therapy in a subject having long COVID-19 or at risk of developing long COVID-19. In these embodiments, the kits comprise one or more antibodies which specifically bind vesicles, one or more antibodies which specifically bind a vesicle biomarker of the disclosure, one or more containers for collecting and or holding the biological sample, and instructions for the kits use.

The present disclosure further provides methods for treating long COVID-19 in a subject. In these embodiments, the disclosure provides methods for treating long COVID-19 in a subject comprising administering an effective amount of an agent to the subject, thereby treating long COVID-19 in the subject.

The section headings are used herein for organizational purposes only, and are not to be construed as in any way limiting the subject matter described herein.

### Biological Sample

The present disclosure provides biomarkers and diagnostic and prognostic methods for long COVID-19. Biomarkers are detected from vesicles (e.g., astrocyte-derived extracellular vesicles and/or neuron-derived extracellular vesicles) from a biological sample obtained from a subject. Biological samples can include any bodily fluid comprising vesicles, including, but not limited to, tissue, whole blood, plasma, serum, lymph, amniotic fluid, and saliva.

In some embodiments, the biological sample of the disclosure can be obtained from blood. In some embodiments, about 1-10 mL of blood is drawn from a subject. In other embodiments, about 10 −50 mL of blood is drawn from a subject. Blood can be drawn from any suitable area of the body, including an arm, a leg, or blood accessible through a central venous catheter. In some embodiments, blood is collected following a treatment or activity. For example, blood can be collected following a medical exam. The timing of collection can also be coordinated to increase the number and/or composition of vesicles present in the sample. For example, blood can be collected following exercise or a treatment that induces vascular dilation.

Blood may be combined with various components following collection to preserve or prepare samples for subsequent techniques. For example, in some embodiments, blood is treated with an anticoagulant, a cell fixative, a protease inhibitor, a phosphatase inhibitor, or preservative(s) for protein or DNA or RNA following collection. In some embodiments, blood is collected via venipuncture using a needle and a syringe that is emptied into collection tubes containing an anticoagulant such as EDTA, heparin, or acid citrate dextrose (ACD). Blood can also be collected using a heparin-coated syringe and hypodermic needle. Blood can also be combined with components that will be useful for cell culture. For example, in some embodiments, blood is combined with cell culture media or supplemented cell culture media (e.g., cytokines). In certain embodiments, platelet-rich plasma (PRP) is mixed with PBS to block ex vivo platelet activation before centrifugation to yield platelet-poor plasma (PPP).

Enrichment and/or Isolation of Neuron-Derived Extracellular Vesicles and Astrocyte-Derived Extracellular Vesicles

Samples can be enriched for neuron-derived extracellular vesicles (NDEV) and/or astrocyte-derived extracellular vesicles (ADEV) through positive selection, negative selection, or a combination of positive and negative selection. In some embodiments, vesicles are directly captured. In other embodiments, blood cells are captured and vesicles are collected from the remaining biological sample.

Samples can also be enriched for vesicles based on the biochemical properties of vesicles. The first step is physical isolation entailing polymer precipitation with centrifugation in one or two cycles. Then, for example, samples can be enriched for vesicles based on differences in antigens. In some of the embodiments, antibody-conjugated magnetic or paramagnetic beads in magnetic field gradients or fluorescently labeled antibodies with flow cytometry are used. In some of the embodiments based on metabolic differences, dye uptake/exclusion measured by flow cytometry or another sorting technology is used. Samples can also be enriched for vesicles based on other biochemical properties known in the art. For example, samples can be enriched for vesicles using ligands or soluble receptors.

In some embodiments, surface markers are used to positively enrich vesicles in the sample. In other embodiments, cell surface markers that are not found on vesicles are used to negatively enrich vesicles by depleting cell populations. Modified versions of flow cytometry sorting may also be used to further enrich for vesicles using surface markers or intracellular or extracellular markers conjugated to fluorescent labels. Intracellular and extracellular markers may include nuclear stains or antibodies against intracellular or extracellular proteins preferentially expressed in vesicles. Cell surface markers may include cell surface antigens that are preferentially expressed on astrocyte-derived vesicles and/or neuron-derived vesicles. In some embodiments, the cell surface marker is an astrocyte-derived vesicle surface marker, including, for example, Glutamine Aspartate Transporter (GLAST). In other embodiments, the vesicle cell-surface marker is CD171 (L1CAM neural adhesion protein). In some embodiments, a monoclonal antibody that specifically binds to GLAST (e.g., ACSA-1, mouse anti-human GLAST antibody) is used to enrich or isolate astrocyte-derived vesicles from the sample. In certain aspects, the antibody against GLAST is biotinylated. In this embodiment, the biotinylated antibody can form an antibody-vesicle complex that can be subsequently isolated using streptavidin-agarose resin or beads. In other embodiments, the antibody is a monoclonal anti-human GLAST antibody (e.g., ACSA-1). Methods useful for isolating vesicles are described in U.S. Pat. Nos. 9,933,440 and 10,393,760.

In other embodiments, vesicles are isolated or enriched from a biological sample comprising: contacting a biological sample with an agent under conditions wherein an vesicle present in said biological sample binds to said agent to form an vesicle-agent complex; and isolating said vesicle from said vesicle-agent complex to obtain a sample containing said vesicle, wherein the purity of the vesicles present in the sample is greater than the purity of vesicles present in the biological sample. In certain embodiments, the contacting is incubating or reacting. In certain embodiments, the vesicles are astrocyte-derived vesicles and/or neuron-derived vesicles. In certain embodiments, the agent is an antibody or a lectin. Lectins useful for forming an vesicle-lectin complex are described in U.S. Patent Application Publication No. 2012/0077263. In some embodiments, multiple isolating or enriching steps are performed. In certain aspects of the present embodiment, a first isolating step is performed to isolate vesicles from a blood sample freed of plasma membrane-derived membrane vesicles and a second isolating step is performed to isolate vesicles from other vesicles. In other embodiments, the vesicle portion of the vesicle-agent complex is lysed using a lysis reagent containing protease and phosphatase inhibitors and the protein levels of the lysed vesicle are assayed. In some embodiments, the antibody-vesicle complex is created on a solid phase. In yet other embodiments, the methods further comprise releasing the vesicle from the antibody-vesicle complex. In certain embodiments, the solid phase is non-magnetic beads, magnetic beads, agarose, or sepharose. In other embodiments, the vesicle is released by exposing the antibody-vesicle complex to low pH between 3.5 and 1.5. In yet other embodiments, the released vesicle is neutralized by adding a high pH solution. In other embodiments, the released vesicles are lysed by incubating the released vesicles with a lysis solution. In still other embodiments, the lysis solution contains inhibitors for proteases and phosphatases.

### Long COVID-19

Although most people with COVID-19 get better within weeks of illness, some people experience post-COVID conditions. Post-COVID conditions are a wide range of new, returning, or ongoing health problems people can experience four or more weeks after first being infected with the virus that causes COVID-19. Even people who did not have COVID-19 symptoms in the days or weeks after they were infected can have post-COVID conditions. These conditions can present as different types and combinations of health problems for different lengths of time.

These post-COVID conditions may also be known as long COVID, long-haul COVID, post-acute COVID-19, long-term effects of COVID, or chronic COVID. Some people experience a range of new or ongoing symptoms that can last weeks or months after first being infected with the virus that causes COVID-19. Unlike some of the other types of post-COVID conditions that tend only to occur in people who have had severe illness, these symptoms can happen to anyone who has had COVID-19, even if the illness was mild, or if they had no initial symptoms. People commonly report experiencing different combinations of the following symptoms: difficulty breathing or shortness of breath, tiredness or fatigue, symptoms that get worse after physical or mental activities (also known as post-exertional malaise), difficulty thinking or concentrating (sometimes referred to as “brain fog”), cough, chest or stomach pain, headache, fast-beating or pounding heart (also known as heart palpitations), joint or muscle pain, pins-and-needles feeling, diarrhea, sleep problems, fever, dizziness on standing (lightheadedness), rash, mood changes, change in smell or taste, and changes in menstrual period cycles.

Some people who had severe illness with COVID-19 experience multiorgan effects or autoimmune conditions over a longer time with symptoms lasting weeks or months after COVID-19 illness. Multiorgan effects can affect many, if not all, body systems, including heart, lung, kidney, skin, and brain functions.

The disclosure provides methods for diagnosing long COVID-19 in a subject and/or identifying a subject at risk of developing long COVID-19, or prescribing a therapeutic regimen or predicting benefit from therapy. Abnormalities were observed in vesicle biomarkers levels from biological samples obtained from COVID-19 subjects, including increased NDEV levels of tethering protein SNPH, which anchors mitochondria to neuron axonal microtubules, as well as dysfunctional NDEV and ADEN % levels of SARS-CoV-2 proteins and mitochondrial proteins (see, e.g., Example 1). Hence, vesicle biomarker abnormalities are associated with development or worsening of long COVID-19. Accordingly, detection of vesicle biomarker abnormalities can be used to identify individuals who will benefit from therapy.

In some embodiments, the subject is a mammalian subject, including, e.g., a cat, a dog, a rodent, etc. In certain embodiments, the subject is a human subject.

In some embodiments, the present disclosure enables a medical practitioner to diagnose or prognose long COVID-19 in a subject. In yet other embodiments, the present disclosure enables a medical practitioner to identify a subject at risk of developing long COVID-19. In other embodiments, the present disclosure enables a medical practitioner to predict whether a subject will later develop long COVID-19. In further embodiments the present disclosure enables a medical practitioner to prescribe a therapeutic regimen or predict benefit from therapy in a subject having long COVID-19 or at risk of developing long COVID-19.

### Biomarkers

Vesicle cargo levels of biomarker proteins are assayed for a subject having or at-risk of having long COVID-19. In some embodiments, one or more SARS-CoV-2 proteins and/or mitochondrial proteins are assayed in order to detect whether or not a subject has or is at risk of developing long COVID-19. In some embodiments, the one or more biomarkers are selected from the group consisting of CD81, syntaphilin (SNPH), subunit 6 of NADH-ubiquinone oxidoreductase (respiratory chain complex I) (CI-6), subunit 10 of cytochrome b-cl oxidase (respiratory chain complex III) (CIII-10), Humanin, myosin VI (MYO6), mitochondrial open-reading frame of the 12S rRNA-c (MOTS-c), voltage-dependent anion-selective channel protein 1 (VDAC1), N-methyl-D-aspartate receptor 1 (NMDAR1), mitochondrial calcium uniporter (MCU), solute carrier family 8 member B1 (SLC24A6) or sodium/calcium exchanger (NCLX), translocator protein (TSOP), leucine zipper EF-hand containing transmembrane 1 protein (LETM1), SARS-CoV-2 S1 (RBD), and/or SARS-CoV-2 nucleocapsid (N) are assayed in order to detect whether or not a subject has or is at risk of developing long COVID-19. As disclosed in Example 2, NDEV levels of the neuroprotective and metabolic regulatory MPs Humanin and MOTS-c and the ion channel VDAC1 were significantly lower and of the axon-toxic NADase SARM1 were significantly higher in acute COVID-19 that progressed to PASC.

One of ordinary skill in the art has several methods and devices available for the detection and analysis of the biomarkers of the instant disclosure. With regard to polypeptides or proteins in patient test samples, immunoassay devices and methods are often used. These devices and methods can utilize labeled molecules in various sandwich, competitive, or non-competitive assay formats, to generate a signal that is related to the presence or amount of an analyte of interest. Additionally, certain methods and devices, such as biosensors and optical immunoassays, may be employed to determine the presence or amount of analytes without the need for a labeled molecule.

Preferably the markers are analyzed using an immunoassay, although other methods are well known to those skilled in the art (for example, the measurement of marker RNA levels). The presence or amount of a marker is generally determined using antibodies specific for each marker and detecting specific binding. Any suitable immunoassay may be utilized, for example, enzyme-linked immunoassays (ELISA), radioimmunoassay (RIAs), competitive binding assays, planar waveguide technology, and the like. Specific immunological binding of the antibody to the marker can be detected directly or indirectly. Direct labels include fluorescent or luminescent tags, metals, dyes, radionuclides, and the like, attached to the antibody. Indirect labels include various enzymes well known in the art, such as alkaline phosphatase, horseradish peroxidase and the like.

The use of immobilized antibodies specific for the biomarkers is also contemplated by the present disclosure. The antibodies could be immobilized onto a variety of solid supports, such as magnetic or chromatographic matrix particles, the surface of an assay place (such as microtiter wells), pieces of a solid substrate material (such as plastic, nylon, paper), and the like. An assay strip could be prepared by coating the antibody or a plurality of antibodies in an array on solid support. This strip could then be dipped into the test sample and then processed quickly through washes and detection steps to generate a measurable signal, such as a colored spot.

The analysis of a plurality of biomarkers may be carried out separately or simultaneously with one test sample. Several biomarkers may be combined into one test for efficient processing of a multiple of samples. In addition, one skilled in the art would recognize the value of testing multiple samples (for example, at successive time points) from the same individual. Such testing of serial samples will allow the identification of changes in marker levels over time. Increases or decreases in biomarker levels, as well as the absence of change in biomarker levels, would provide useful information about disease status that includes, but is not limited to the appropriateness of drug therapies, the effectiveness of various therapies, identification of the severity of long COVID-19, susceptibility to long COVID-19, and prognosis of the patient's outcome, including risk of development of long COVID-19.

An assay consisting of a combination of the biomarkers referenced in the instant disclosure may be constructed to provide relevant information related to differential diagnosis. Such a panel may be constructed using 1, 2, 3, 4, 5, 6, 7, 8, 9, 10, 15, 20, or more individual markers. The analysis of a single biomarker or subsets of biomarkers comprising a larger panel of biomarkers could be carried out using methods described within the instant disclosure to optimize clinical sensitivity or specificity in various clinical settings.

The analysis of markers could be carried out in a variety of physical formats as well. For example, the use of microtiter plates or automation could be used to facilitate the processing of large numbers of test samples. Alternatively, single sample formats could be developed to facilitate immediate treatment and diagnosis in a timely fashion, for example, in ambulatory transport or emergency room settings. Particularly useful physical formats comprise surfaces having a plurality of discrete, addressable locations for the detection of a plurality of different analytes. Such formats include protein microarrays, or “protein chips” and capillary devices.

Biomarkers of the present disclosure serve an important role in the early detection and monitoring of long COVID-19. Biomarkers are typically substances found in a bodily sample that can be measured. The measured amount can correlate with underlying disorder or disease pathophysiology and probability of developing long COVID-19 in the future. In patients receiving treatment for their condition, the measured amount will also correlate with responsiveness to therapy.

In some embodiments, the biomarker is measured by a method selected from the group consisting of immunohistochemistry, immunocytochemistry, immunofluorescence, immunoprecipitation, western blotting, and ELISA.

### Clinical Assay Performance

The methods of the present disclosure for detecting long COVID-19 may be used in clinical assays to diagnose or prognose long COVID-19 in a subject, identify a subject at risk of long COVID-19, and/or for prescribing a therapeutic regimen or predicting benefit from therapy in a subject having long COVID-19. Clinical assay performance can be assessed by determining the assay's sensitivity, specificity, area under the ROC curve (AUC), accuracy, positive predictive value (PPV), and negative predictive value (NPV). Disclosed herein are assays for diagnosing or prognosing long COVID-19 in a subject, identifying a subject at risk of long COVID-19, or for prescribing a therapeutic regimen or predicting benefit from therapy in a subject having long COVID-19.

The clinical performance of the assay may be based on sensitivity. The sensitivity of an assay of the present disclosure may be at least about 40%, 45%, 50%, 55%, 60%, 65%, 70%, 75%, 80%, 85%, 90%, 95%, 99%, or 100%. The clinical performance of the assay may be based on specificity. The specificity of an assay of the present disclosure may be at least about 40%, 45%, 50%, 55%, 60%, 65%, 70%, 75%, 80%, 85%, 90%, 95%, 99%, or 100%. The clinical performance of the assay may be based on area under the ROC curve (AUC). The AUC of an assay of the present disclosure may be at least about 0.5, 0.55, 0.6, 0.65, 0.7, 0.75, 0.8, 0.85, 0.9, or 0.95. The clinical performance of the assay may be based on accuracy. The accuracy of an assay of the present disclosure may be at least about 40%, 45%, 50%, 55%, 60%, 65%, 70%, 75%, 80%, 85%, 90%, 95%, 99%, or 100%.

### Agents and Compositions

Agents and compositions useful in the methods of the present disclosure include agents and compositions that specifically recognize one or more vesicle biomarkers associated with long COVID-19, including a SARS-CoV-2 protein and/or mitochondrial protein or any combination thereof. In some embodiments, the one or more vesicle biomarker is CD81, syntaphilin (SNPH), subunit 6 of NADH-ubiquinone oxidoreductase (respiratory chain complex I) (CI-6), subunit 10 of cytochrome b-cl oxidase (respiratory chain complex III) (CIII-10), Humanin, myosin VI (MYO6), mitochondrial open-reading frame of the 12S rRNA-c (MOTS-c), voltage-dependent anion-selective channel protein 1 (VDAC1), N-methyl-D-aspartate receptor 1 (NMDAR1), mitochondrial calcium uniporter (MCU), solute carrier family 8 member B1 (SLC24A6) or sodium/calcium exchanger (NCLX), translocator protein (TSOP), leucine zipper EF-hand containing transmembrane 1 protein (LETM1), SARS-CoV-2 S1 (RBD), and/or SARS-CoV-2 nucleocapsid (N) or any combination thereof. In some embodiments, the agent or composition enhances the activity of at least one biomarker. In other embodiments, the agent or composition decreases the activity of at least one biomarker. In some embodiments, the agent composition increases the levels of at least one biomarker in the subject. In other embodiments, the agent composition decreases the levels of at least one biomarker in the subject. In yet other embodiments, the agent composition comprises a peptide, a nucleic acid, an antibody, or a small molecule.

In certain embodiments, the present disclosure relates to agents and/or compositions that specifically detect a biomarker associated with long COVID-19. As detailed elsewhere herein, the disclosure is based upon the finding that SARS-CoV-2 protein and/or mitochondrial protein are specific biomarkers for long COVID-19. In one embodiment, the compositions of the disclosure specifically bind to and detect one or more of the following biomarkers: CD81, syntaphilin (SNPH), subunit 6 of NADH-ubiquinone oxidoreductase (respiratory chain complex I) (CI-6), subunit 10 of cytochrome b-cl oxidase (respiratory chain complex III) (CIII-10), Humanin, myosin VI (MYO6), mitochondrial open-reading frame of the 12S rRNA-c (MOTS-c), voltage-dependent anion-selective channel protein 1 (VDAC1), N-methyl-D-aspartate receptor 1 (NMDAR1), mitochondrial calcium uniporter (MCU), solute carrier family 8 member B1 (SLC24A6) or sodium/calcium exchanger (NCLX), translocator protein (TSOP), leucine zipper EF-hand containing transmembrane 1 protein (LETM1), SARS-CoV-2 S1 (RBD), and/or SARS-CoV-2 nucleocapsid (N), or any combination thereof. The agent and/or composition of the disclosure can comprise an antibody, a peptide, a small molecule, a nucleic acid, and the like.

In some embodiments, the agent and/or composition comprises an antibody, wherein the antibody specifically binds to a biomarker or vesicle. The term “antibody” as used herein and further discussed below is intended to include fragments thereof which are also specifically reactive with a biomarker or vesicle (e.g., exosome). Antibodies can be fragmented using conventional techniques and the fragments screened for utility in the same manner as described above for whole antibodies. For example, F(ab)₂ fragments can be generated by treating antibody with pepsin. The resulting F(ab)₂ fragment can be treated to reduce disulfide bridges to produce Fab fragments. Antigen-binding portions may also be produced by recombinant DNA techniques or by enzymatic or chemical cleavage of intact antibodies. Antigen-binding portions include, inter alia, Fab, Fab′, F(ab′)₂, Fv, dAb, and complementarity determining region (CDR) fragments, single-chain antibodies (scFv), single domain antibodies, bispecific antibodies, chimeric antibodies, humanized antibodies, diabodies and polypeptides that contain at least a portion of an immunoglobulin that is sufficient to confer specific antigen binding to the polypeptide. In certain embodiments, the antibody further comprises a label attached thereto and able to be detected (e.g., the label can be a radioisotope, fluorescent compound, enzyme or enzyme co-factor).

In certain embodiments, an antibody of the disclosure is a monoclonal antibody, and in certain embodiments, the disclosure makes available methods for generating novel antibodies that specifically bind the biomarker or the vesicle of the disclosure. For example, a method for generating a monoclonal antibody that specifically binds a biomarker or vesicle, may comprise administering to a mouse an amount of an immunogenic composition comprising the biomarker or vesicle, or fragment thereof, effective to stimulate a detectable immune response, obtaining antibody-producing cells (e.g., cells from the spleen) from the mouse and fusing the antibody-producing cells with myeloma cells to obtain antibody-producing hybridomas, and testing the antibody-producing hybridomas to identify a hybridoma that produces a monocolonal antibody that binds specifically to the biomarker or vesicle. Once obtained, a hybridoma can be propagated in a cell culture, optionally in culture conditions where the hybridoma-derived cells produce the monoclonal antibody that binds specifically to the biomarker or vesicle. The monoclonal antibody may be purified from the cell culture.

The term “specifically reactive with” or “specifically binds” as used in reference to an antibody is intended to mean, as is generally understood in the art, that the antibody is sufficiently selective between the antigen of interest (e.g., a biomarker or vesicle) and other antigens that are not of interest. In certain methods employing the antibody, such as therapeutic applications, a higher degree of specificity in binding may be desirable. Monoclonal antibodies generally have a greater tendency (as compared to polyclonal antibodies) to discriminate effectively between the desired antigens and cross-reacting polypeptides. One characteristic that influences the specificity of an antibody:antigen interaction is the affinity of the antibody for the antigen. Although the desired specificity may be reached with a range of different affinities, generally preferred antibodies will have an affinity (a dissociation constant) of about 10⁻⁶, 10⁻⁷, 10⁻⁸, 10⁻⁹ or less.

Antibodies can be generated to bind specifically to an epitope of a vesicle or a biomarker of the present disclosure, including, for example, vesicle surface markers, such as Glutamine Aspartate Transporter (GLAST) or CD171 (L1CAM neural adhesion protein).

In addition, the techniques used to screen antibodies in order to identify a desirable antibody may influence the properties of the antibody obtained. A variety of different techniques are available for testing interaction between antibodies and antigens to identify particularly desirable antibodies. Such techniques include ELISAs, surface plasmon resonance binding assays (e.g., the Biacore binding assay, Biacore AB, Uppsala, Sweden), sandwich assays (e.g., the paramagnetic bead system of IGEN International, Inc., Gaithersburg, Md.), western blots, immunoprecipitation assays, immunocytochemistry, and immunohistochemistry.

In some embodiments, the present disclosure relates to agent and/or compositions used for treating or preventing long COVID-19. As detailed elsewhere herein, abnormal levels of vesicle biomarkers are implicated in the pathology of long COVID-19 (see, e.g., Example 1). Therefore, in one embodiment, the present disclosure provides compositions that inhibit or reduce abnormalities in levels of vesicle biomarkers. Compositions and agents useful for preventing and/or reducing abnormalities in levels of vesicle biomarkers may include proteins, peptides, nucleic acids, small molecules, and the like.

### Methods of Treatment

The present disclosure provides methods for treating long COVID-19 in a subject, the method comprising administering an effective amount of an agent to a subject having or suspected of having long COVID-19, thereby treating the long COVID-19.

Generally, the therapeutic/diagnostic agents used in the disclosure are administered to a subject in an effective amount. Generally, an effective amount is an amount effective to (1) reduce the symptoms of the long COVID-19 to be treated, (2) induce a pharmacological change relevant to treating the long COVID-19 to be treated or (3) detect long COVID-19 in vivo or in vitro. For example, an effective amount of an agent of the disclosure includes an amount effective to: prevent or reduce difficulty breathing or shortness of breath, tiredness or fatigue, symptoms that get worse after physical or mental activities (also known as post-exertional malaise), difficulty thinking or concentrating (sometimes referred to as “brain fog”), cough, chest or stomach pain, headache, fast-beating or pounding heart (also known as heart palpitations), joint or muscle pain, pins-and-needles feeling, diarrhea, sleep problems, fever, dizziness on standing (lightheadedness), rash, mood changes, change in smell or taste, and changes in menstrual period cycles.

Effective amounts of the agents can be any amount or dose sufficient to bring about the desired effect and will depend, in part, on the condition, type of long COVID-19, the size and condition of the patient, as well as other factors readily known to those skilled in the art. The dosages can be given as a single dose, or as several doses, for example, divided over the course of several weeks. It is specifically contemplated that therapeutic agent may need to be administered immediately (or as soon as possible) and for several months following long COVID-19 diagnosis to be optimally effective.

The disclosure is also directed toward methods of treatment utilizing the therapeutic compositions of the present disclosure. The method comprises administering the therapeutic agent to a subject in need of such administration, such as, for example, a subject with long COVID-19.

The therapeutic agents of the instant disclosure can be administered by any suitable means as described herein, including, for example, parenteral, topical, oral or local administration, such as intradermally, by injection, or by aerosol. In one embodiment of the disclosure, the agent is administered by injection. Such injection can be locally administered to any affected area. A therapeutic composition can be administered in a variety of unit dosage forms depending upon the method of administration. For example, unit dosage forms suitable for oral administration of a subject include powder, tablets, pills and capsules. Preferred delivery methods for a therapeutic composition of the disclosure include intravenous administration and local administration by, for example, injection or topical administration. For particular modes of delivery, a therapeutic composition of the disclosure can be formulated in an excipient of the disclosure. A therapeutic agent of the disclosure can be administered to any subject, to mammals, and to humans.

The particular mode of administration will depend on the long COVID-19 pathology to be treated. It is contemplated that administration of the agents of the present disclosure may be via any bodily fluid, or any target or any tissue accessible through a body fluid.

Furthermore, the methods of the disclosure can be used for monitoring the efficacy of therapy in a patient. The method comprises: analyzing the levels of one or more biomarkers selected from the group consisting of a SARS-CoV-2 protein and/or mitochondrial protein from vesicles from biological samples from the patient before and after the patient undergoes the therapy, in conjunction with respective reference levels for the biomarkers. Abnormal vesicle levels of SARS-CoV-2 proteins and/or mitochondrial proteins correlate with long COVID-19 severity and indicate that the patient is worsening or not responding to the therapy, and normalizing vesicle levels of SARS-CoV-2 protein and/or mitochondrial protein correlate with reduced long COVID-19 severity and indicate that the condition of the patient is improving.

In some embodiments, the methods of the disclosure provide a method for treating long COVID-19 the method comprising the steps of: obtaining a biological sample from a subject suspected of having long COVID-19, wherein the sample comprises vesicles; measuring the level of one or more biomarkers selected from the group consisting of a SARS-CoV-2 protein and/or mitochondrial protein from the biological sample, wherein an altered level of the one or more biomarkers in the sample relative to the level in a control sample is indicative of a need for treatment; and administering an effective amount of an agent to the subject thereby treating long COVID-19 in the subject. In some embodiments, the biomarker comprises CD81, syntaphilin (SNPH), subunit 6 of NADH-ubiquinone oxidoreductase (respiratory chain complex I) (CI-6), subunit 10 of cytochrome b-cl oxidase (respiratory chain complex III) (CIII-10), Humanin, myosin VI (MYO6), mitochondrial open-reading frame of the 12S rRNA-c (MOTS-c), voltage-dependent anion-selective channel protein 1 (VDAC1), N-methyl-D-aspartate receptor 1 (NMDAR1), mitochondrial calcium uniporter (MCU), solute carrier family 8 member B1 (SLC24A6) or sodium/calcium exchanger (NCLX), translocator protein (TSOP), leucine zipper EF-hand containing transmembrane 1 protein (LETM1), SARS-CoV-2 S1 (RBD), and/or SARS-CoV-2 nucleocapsid (N).

### Kits

Another aspect of the disclosure encompasses kits for detecting or monitoring long COVID-19 in a subject. A variety of kits having different components are contemplated by the current disclosure. Generally speaking, the kit will include the means for quantifying one or more biomarkers in a subject. In another embodiment, the kit will include means for collecting a biological sample, means for quantifying one or more biomarkers in the biological sample, and instructions for use of the kit contents. In certain embodiments, the kit comprises a means for enriching or isolating vesicles in a biological sample. In some embodiments, the vesicles are astrocyte-derived extracellular vesicles and/or neuron-derived extracellular vesicles. In further aspects, the means for enriching or isolating vesicles comprises reagents necessary to enrich or isolate vesicles from a biological sample. In certain aspects, the kit comprises a means for quantifying the amount of a biomarker. In further aspects, the means for quantifying the amount of a biomarker comprises reagents necessary to detect the amount of a biomarker. In some aspects, the biomarker is a SARS-CoV-2 protein and/or mitochondrial protein. In other aspects, the biomarker is CD81, syntaphilin (SNPH), subunit 6 of NADH-ubiquinone oxidoreductase (respiratory chain complex I) (CI-6), subunit 10 of cytochrome b-cl oxidase (respiratory chain complex III) (CIII-10), Humanin, myosin VI (MYO6), mitochondrial open-reading frame of the 12S rRNA-c (MOTS-c), voltage-dependent anion-selective channel protein 1 (VDAC1), N-methyl-D-aspartate receptor 1 (NMDAR1), mitochondrial calcium uniporter (MCU), solute carrier family 8 member B1 (SLC24A6) or sodium/calcium exchanger (NCLX), translocator protein (TSOP), leucine zipper EF-hand containing transmembrane 1 protein (LETM1), SARS-CoV-2 S1 (RBD), and/or SARS-CoV-2 nucleocapsid (N).

These and other embodiments of the present disclosure will readily occur to those of ordinary skill in the art in view of the disclosure herein.

### EXAMPLES

The disclosure will be further understood by reference to the following examples, which are intended to be purely exemplary of the disclosure. These examples are provided solely to illustrate the claimed disclosure. The present disclosure is not limited in scope by the exemplified embodiments, which are intended as illustrations of single aspects of the disclosure only. Any methods that are functionally equivalent are within the scope of the disclosure. Various modifications of the disclosure in addition to those described herein will become apparent to those skilled in the art from the foregoing description. Such modifications are intended to fall within the scope of the appended claims.

### Example 1: Detection of SARS-CoV-2 Proteins and Mitochondrial Proteins in Neuron-Derived Extracellular Vesicles (NDEVs) and Astrocyte-Derived Extracellular Vesicles (ADEVs) in Biological Samples from COVID-19 Patients

SARS-CoV-2 proteins and mitochondrial proteins in plasma neuron-derived extracellular vesicles (NDEVs) and astrocyte-derived extracellular vesicles (ADEVs) were quantified in biological samples from COVID-19 patients as follows. Participants were enrolled in the San Francisco-based Long-term Impact of Infection with Novel Coronavirus (LIINC) COVID-19 recovery cohort (NCT04362150; www.liincstudy.org) (Peluso et al. medRxiv 2021). Their evaluation included confirmation of prior SARS-CoV-2 nucleic acid positivity, detailed assessment of acute and post-acute COVID-19-attributed symptoms and a complete range of basic clinical laboratory studies. At each visit, participants completed a general Patient Health Questionnaire (PHQ) and several other specific questionnaires regarding current symptoms. From these data, relevant neuropsychiatric manifestations were tabulated: irritability or agitation from the PHQ and moderate or severe anxiety or depression from EuroQuol, PHQ-8 and General Anxiety Disorder-7 (GAD-7) questionnaires (Rabin et al. Ann Med 2001; 33: 337-43, Kroenke et al. J Affect Disord 2009; 114: 163-73, and Spitzer et al. Arch Intern Med 2006; 166: 1092-7). From this clinical information and lab data, four COVID-19 groups were assembled, in addition to age- and sex-matched never-infected controls (Ctl): convalescence from SARS-CoV-2 infection with no evidence of residual infection or PASC, PASC without (w/o) neuropsychiatric (NP) symptoms, PASC with (w/) NP symptoms (<7 symptoms) and PASC w/severe NP symptoms (>7 symptoms) (Table 1).

Isolation of Neuron-Derived Extracellular Vesicles (NDEVs) and Astrocyte-Derived Extracellular Vesicles (ADEVs).

Portions of 0.25 ml of thawed EDTA-plasma from the PASC period of each group (Table 1) were incubated with thromboplastin D (ThermoFisher Scientific, Waltham, Mass.) and treated with protease inhibitor cocktail (Roche, Indianapolis, Ind.) and phosphatase inhibitor cocktail (Thermo Fisher Scientific; DBS′) as described (Goetzl et al. Faseb J 2018; 32: 888-93). After centrifugation at 3000×g for 30 minutes at 4° C., total extracellular vesicles (EVs) were harvested from resultant supernatants by precipitation with 126 μL per tube of ExoQuick (System Biosciences, Mountain View, Calif.) and centrifugation at 1500×g for 30 minutes at 4° C.

TABLE 1
Demographic and clinical characteristics of participant groups

| | | | | | PASC w/ |
|---|---|---|---|---|---|
| | | COVID-19 w/o | PASC w/o | PASC w/ | severe |
| | Control | PASC | NP | NP | NP |
| Number | 12 | 8 | 15 | 15 | 8 |
| Mean age (range) | 48 (26-67) | 42 (30-54) | 46 (34-56) | 34 (31-46) | 49 (44-52) |
| Female/male/TG male | 9/3/0 | 8/0/0 | 9/6/0 | 9/6/0 | 7/0/1 |
| Race, ethnicity | 4/3/1/4/0 | 2/5/0/1/0 | snimix | 3/10/1/0/1 | 3/5/0/0/0 |
| H/W/B/A/O | | | | | |
| Co-Morbidities | | | | | |
| Autoimmune disease | 3 | 0 | 3 | 2 | 1 |
| Cancer | 1 | 1 | 0 | 0 | 1 |
| Diabetes | 1 | 0 | 0 | 0 | 0 |
| Cardiovascular disease | 2 | 0 | 1 | 0 | 1 |
| HIV/AIDS | 0 | 0 | 0 | 0 | 0 |
| Lung disease | 0 | 1 | 0 | 4 | 2 |
| Kidney disease | 1 | 0 | 0 | 0 | 0 |
| Days from symptom | N/A | 57 (35-64) | 62 (35-81) | 60 (37-80) | 60 (44-84) |
| onset to blood | | | | | |
| sampling, mean (range) | | | | | |
| Manifestations of PASC | | | | | |
| Total symptoms at time | 0 | 0 | 0-9 | 0-7 | 8-13 |
| of blood sampling | | | | | |
| Feeling fatigue or low | 0 | 0 | 5 | 3 | 8 |
| energy | | | | | |
| Trouble w/smell or | 0 | 0 | 4 | 5 | 3 |
| taste | | | | | |
| Muscle aches | 0 | 0 | 2 | 4 | 6 |
| Loss of appetite | 0 | 0 | 0 | 2 | 4 |
| Trouble concentrating | 0 | 0 | 0 | 6 | 8 |
| or thinking | | | | | |
| Trouble w/vision | 0 | 0 | 1 | 1 | 3 |
| Trouble w/balance | 0 | 0 | 1 | 2 | 5 |
| Trouble w/sleeping | 0 | 0 | 1 | 6 | 5 |
| Cough | 0 | 0 | 1 | 2 | 1 |
| Shortness of breath | 0 | 0 | 5 | 3 | 6 |
| Chest pain | 0 | 0 | 1 | 2 | 3 |

H, Hispanic;
W, white;
B, black;
A, Asian;
O, other;
TG, transgenic;
np, neuropsychiatric manifestations;
w/, with;
w/o, without.

To enrich NDEVs including neuron-derived exosomes (NDEs), replicate preparations of total plasma EVs were resuspended in 0.35 mL of DBS' with 2.0 μg of mouse anti-human CD171 (L1CAM neural adhesion protein) biotinylated antibody (clone 5G3; eBiosciences, San Diego, Calif.) in 50 μL of 3% bovine serum albumin (BSA; 1:3.33 dilution of Blocker BSA 10% solution in DBS; ThermoFisher Scientific) per tube. After this incubation 10 μL of streptavidin-agarose Ultralink resin (ThermoFisher Scientific) in 40 μL of 3% BSA were added to each tube followed by a second incubation as described in Goetzl et al. Ann Neurol 2018; 83: 544-52. After centrifugation at 800×g for 10 minutes at 4° C. and removal of the supernatant, each pellet with NDEVs was suspended in 100 μL of cold 0.05 M glycine-HCl (pH 3.0) for 5 minutes and centrifuged at 4000×g for 10 minutes, all at 4° C. Glycine-HCl supernatants then were transferred to clean tubes containing 25 μL of 10% BSA and 10 μL of 1 M Tris-HCl (pH 8.0) and mixed gently. An aliquot of 5 μL was removed from each tube for NEV counts before addition of 370 μL of mammalian protein extraction reagent (M-PER, ThermoFisher Scientific). Astrocyte-derived EVs (ADEVs) were enriched from total plasma EVs by immunoabsorption with mouse anti-human glutamine aspartate transporter (GLAST) (ACSA-1) biotinylated antibody (Miltenyi Biotec, Inc., Auburn, Calif., USA), that yielded numbers, sizes, and contents of exosome markers, glial fibrillary acidic protein (GFAP) and glial-derived neurotrophic factor (GDNF) identical to those described in Goetzl et al. Faseb J 2016; 30: 3853-9 and Goetzl et al. Ann Neurol 2018; 83: 544-52. Resultant 0.5 mL lysates of NDEVs were frozen and thawed twice, and then stored at −80° C.

For counting and sizing of extracellular vesicles, each suspension was diluted 1:50 in PBS. The mean diameter (nanometers) and concentration (particles per milliliter) of EVs in each suspension were determined by nanoparticle tracking analysis (NTA) using the Nanosight NS500 system with a G532 nm laser module and NTA 3.1 nanoparticle tracking software (Malvern Instruments, Malvern, United Kingdom) as described in Mustapic et al. Front Neurosci 2017; 11: 278.

Quantification of NDEV and ADEV Proteins

Human NDEV and ADEV proteins were measured and quantified by enzyme-linked immunosorbent assay (ELISA) kits for tetraspanning exosome marker CD81, subunit 6 of NADH-ubiquinone oxidoreductase (respiratory chain complex I) (CI-6), 16S rRNA-encoded Humanin (CUSABIO by American Research Products, Waltham, Mass.), N-methyl-D-aspartate receptor 1 (NMDAR1) (Novus Biologicals, LLC, Centennial, CO), myosin VI (MYO6), subunit 10 of cytochrome b-cl oxidase (respiratory chain complex III) (CIII-10) (Abbkine Scientific Co., Ltd. by American Research Products), mitochondrial open-reading frame of the 12S rRNA-c (MOTS-c) (Cloud-Clone Corp. by American Research Products), SARS-CoV-2 protein 51 (RBD), SARS-CoV-2 protein N (Abcam, Inc., Waltham, Mass. and Raybiotech Life, Inc., Peachtree Corners, GA), syntaphilin (SNPH), translocator protein (TSPO), leucine zipper EF-hand containing transmembrane 1 protein (LETM1), voltage-dependent anion-selective channel protein 1 (VDAC1), mitochondrial calcium uniporter protein (MCU), and solute carrier family 8 member B1 (SLC24A6) or mitochondrial Na+/Ca⁺⁺ exchanger (NCLX) (Wuhan FineTest Biotech Co. by American Research Products and Abbexa, Ltd., Cambridge, UK). The mean value for all determinations of CD81 in each assay group was set at 1.00, and relative values of CD81 for each sample were used to normalize their recovery. All ELISAs were performed by one investigator (EJG) without knowledge of the identity of any subject.

After establishing that data were normally distributed, the significance of differences between COVID-19 patient levels and control levels were calculated by an unpaired t test (Graphpad Holdings, LLC, San Diego, Calif.). Pearson Correlation Coefficient analysis was performed to assess relationships between NDEV counts and NDEV levels of CD81 (Graphpad Prism 9). There were no adjustments for potential confounding variables.

The participants in each sub-group were predominantly early middle age, female and of Hispanic and European Caucasian ethnicity (Table 1). None of the participants with convalescent acute COVID-19 w/o post-acute sequelae of SARS-CoV-2 infection (PASC) had respiratory or neuropsychiatric (NP) abnormalities. Of the subjects with PASC, 30 had mild to moderate disease and 8 had severe disease. There was a higher prevalence of pre-existing lung disease in the participants who had PASC with NP (PASC w/NP) and respectively lower and higher aggregate incidences of co-morbidities in the convalescent COVID-19 w/o PASC and healthy control (Ctl) sub-groups. No participant had HIV/AIDS and none had received any SARS-CoV-2 vaccine. The prevalence and severity of respiratory-type abnormalities were consistent with similarly mild to moderate involvement in the PASC without NP manifestations (PASC w/o NP) and PASC with NP sub-groups (Table 1). None of the PASC w/o NP participants had trouble concentrating or thinking and none had new problems with memory, vision, balance, sleeping or appetite.

EV Properties

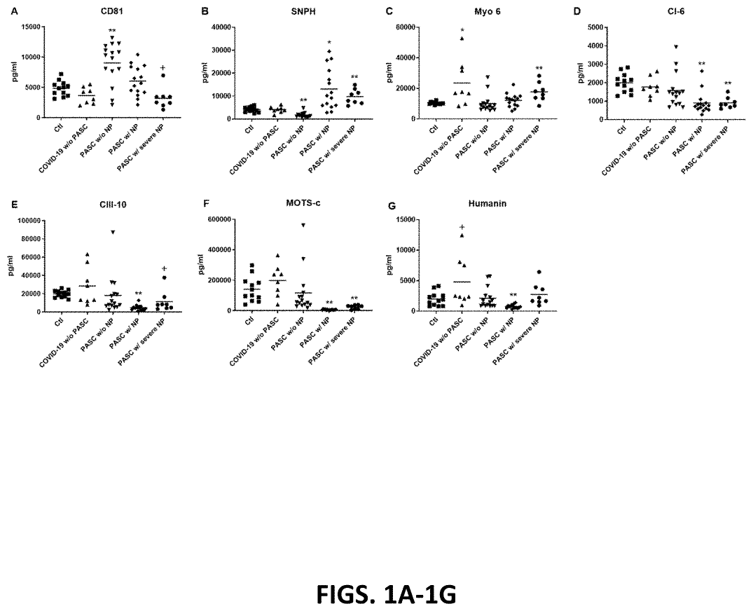

Counts of NDEVs in preparations from the PASC w/o NP sub-group (15.2±1.35×10¹⁰/ml) and the PASC with severe NP sub-group (6.25±0.94×10¹⁰/ml) were respectively significantly higher (p=0.0002) and lower (p=0.0235) than in Ctls (8.51±0.41×10¹⁰/ml) and paralleled the levels for the exosome marker CD81 (FIG. 1A). Counts of NDEVs in preparations from the PASC w/o NP sub-group were significantly higher (p=0.0078) than those of the PASC w/NP subgroup (10.7±0.86×10¹⁰/ml), as were the levels of CD81 (FIG. 1A). The Pearson Correlation Coefficient for the comparison of NDEV counts with CD81 levels of the total group was 0.9879 (p<0.0001). CD81 levels thus were used to normalize levels of all analytes as described in Mustapic et al. Front Neurosci 2017; 11: 278. All counts of ADEVs were in the range of 6.72±0.40×10¹⁰/m1 to 8.86±0.59×10¹⁰/m1 with no significant differences among sub-groups.

NDEV and ADEV Contents of SARS-CoV-2 Proteins

NDEV and ADEV levels of SARS-CoV-2 proteins spike S1 with the cellular receptor binding domain (RBD) and nucleocapsid (N) were significantly higher in all affected sub-groups than in the healthy controls (Table 2). Comparing protein S1 levels among non-control groups revealed a significant difference only between convalescent COVID-19 w/o PASC and PASC w/NP (p=0.0172) for NDEVs, whereas there was a significant difference between COVID-19 w/o PASC and all three PASC sub-groups (p<0.01) for ADEVs. Comparing protein N levels among non-control groups revealed significant differences between PASC w/NP and PASC w/o NP (p=0.0298 for NDEVs and p=0.0228 for ADEVs), and between COVID-19 w/o PASC and both PASC w/NP (p=0.0109 for NDEVs and p=0.0029 for ADEVs) and PASC w/severe NP (p=0.0212 for NDEVs and p=0.0109 for ADEVs). The mean levels of both S1 (RBD) and N proteins were significantly higher in NDEVs than ADEVs of the PASC w/NP subgroup, which implied greater invasion of neuronal than astrocytic mitochondria by SARS-CoV-2 and suggested the importance of determining levels of NDEV mitochondrial proteins (MPs) known to be abnormal in several central nervous system diseases.

TABLE 2
SARS-CoV-2 proteins in ADEVs and NDEVs of participant groups

| | SARS- | | | | | PASC w/ |
|---|---|---|---|---|---|---|
| Source | CoV-2 | Control | COVID-19 w/o | PASC w/o NP | PASC w/NP | severe NP |
| of EVs | protein | (n = 12) | PASC (n = 8) | (n = 15) | (n = 15) | (n = 8) |
| NDEVs | S1 | 197 ± 11.2 | 628 ± 80.8** | 839 ± 217^{†} | 1128 ± 133** | 994 ± 154** |
| | (RBD) | | | | | |
| | N | 936 ± 90.9 | 11,265 ± 2495** | 15,283 ± 3308** | 26,838 ± 3814** | 23,560 ± 4027** |
| ADEVs | S1 | 84.2 ± 10.1 | 371 ± 50.9** | 727 ± 80.2** | 714 ± 60.9** | 645 ± 66.6** |
| | (RBD) | | | | | |
| | N | 371 ± 13.8 | 8576 ± 2332** | 12,452 ± 1627** | 17,979 ± 1618** | 17,241 ± 1810** |

N = nucleocapsid protein, NP = neuropsychiatric manifestations, RBD = receptor-binding domain, w/= with, w/o = without. Each value is the mean pg/ml ± SEM. Statistical significance of differences between each value and the control value were determined by an unpaired Student's t test: ^{†}, p < 0.05; **, p < 0.001. Comparing protein S₁ levels among non-control groups revealed a significant difference only between convalescent COVID-19 w/o PASC and PASC w/NP (p = 0.0172) for NDEVs, whereas there was a significant difference between COVID-19 w/o PASC and all three PASC sub-groups (p < 0.01) for ADEVs. Comparing protein N levels among non-control groups revealed significant differences between PASC w/NP and PASC w/o NP (p = 0.0298 for NDEVs and p = 0.0228 for ADEVs), and between COVID-19 w/o PASC and both PASC w/NP (p = 0.0109 for NDEVs and p = 0.0029 for ADEVs) and PASC w/severe NP (p = 0.0212 for NDEVs and p = 0.0109 for ADEVs).

NDEV Levels of Mitochondrial Proteins (MPs)

Levels of three pairs of mitochondrial proteins (MPs) in plasma NDEV extracts have been shown to be abnormal relative to those of matched healthy controls in subjects with Alzheimer's disease (AD), major depressive disorders (MDDs) and first episode of psychosis (FP) in early schizophrenia (Goetzl et al. Transl Psychiatry 2020; 10: 361, Goetzl et al. Mol Psychiatry 2021, and Yao et al. Biomedicines 2021; 9). These MPs were quantified initially in the NDEVs of current participants to identify effects of SARS-CoV-2 invasion of neurons. The NDEV level of tethering protein SNPH, which anchors mitochondria to neuron axonal microtubules, was statistically significantly decreased relative to that of Ctls in PASC w/o NP as in MDD, increased in PASC w/NP and PASC w/severe NP as in FP, but unaffected in convalescent COVID-19 w/o PASC (FIG. 1B). The NDEV level of tethering protein Myo 6, which anchors mitochondria to synaptic microfilaments, was significantly increased relative to that of Ctls only in PASC w/severe NP and convalescent COVID-19 w/o PASC (FIG. 1C). NDEV levels of constituent proteins CI-6 (FIG. 1D) and CIII-10 (FIG. 1E) of the inner mitochondrial membrane electron transfer chain both were significantly decreased relative to Ctls in PASC w/NP and PASC w/severe NP as in MDDs and FP, but unchanged in PASC w/o NP and convalescent COVID-19 w/o PASC. The NDEV level of metabolic regulatory peptide MOTS-c, which is encoded by a mitochondrial ribosomal RNA, also was decreased significantly relative to Ctls in PASC w/NP and PASC w/severe NP as in MDDs and FP, but unchanged in PASC w/o NP and convalescent COVID-19 w/o PASC (FIG. 1F). The NDEV level of neuroprotective MPs Humanin, which is encoded by another mitochondrial ribosomal RNA, was decreased significantly relative to Ctls in PASC w/NP as in MDDs and FP, but unchanged in PASC w/o NP and increased marginally relative to Ctls in convalescent COVID-19 w/o PASC (FIG. 1G).

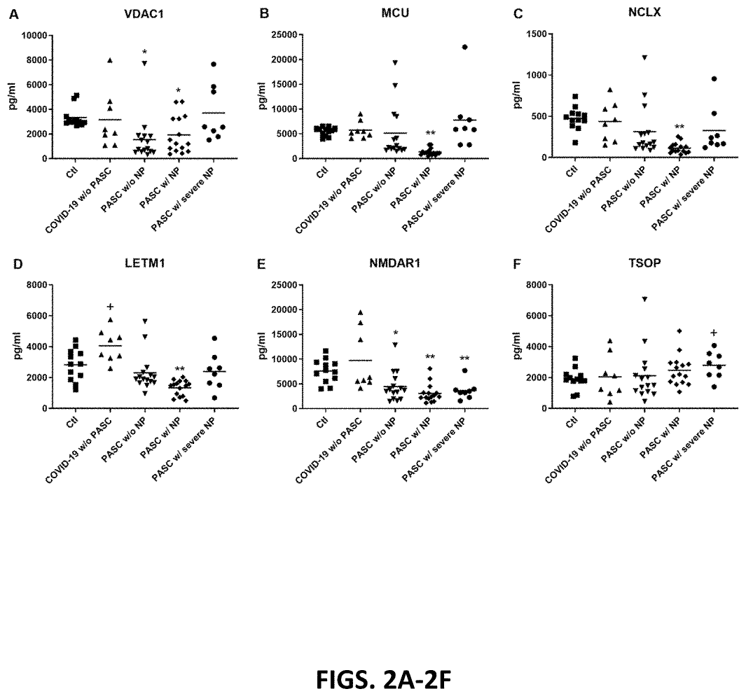

NDEV levels of six neuronal mitochondrial membrane ion channel/exchanger or transporter proteins, that have not been examined previously and of which five are involved in Ca′ movement across the mitochondrial inner (IMM) or outer (OMM) membranes, were quantified for the same four groups of patients and Ctls (FIG. 2). VDAC1, the quantitatively predominant protein in OMM, is a voltage-gated channel for Ca⁺⁺, other ions, diverse metabolites and nucleotides (Camara et al. Front Physiol 2017; 8: 460). NMDAR1 in the IMM of synaptic and extrasynaptic mitchondria, as well as in neuronal plasma membranes, is a ligand-gated Ca′ channel (Korde et al. J Biol Chem 2012; 287: 35192-200). VDAC1 and NMDAR1 levels in NDEV extracts both were significantly depressed below those of Ctls in PASC w/o NP and PASC w/NP, but not in convalescent COVID-19 w/o PASC (FIGS. 2A and 2E). In contrast, NDEV levels of the mechanistically distinct MCU, NCLX and LETM1 IMM channels for Ca⁺⁺ all were decreased very significantly below those of Ctls in PASC w/NP, but not in PASC w/o NP or convalescent COVID-19 w/o PASC (FIGS. 2B, C and D). The NDEV levels of OMM cholesterol translocator TSOP showed no differences from those of Ctls except for a marginal increase in PASC w/severe NP (FIG. 2F).

Ca⁺⁺ concentration in the mitochondrial matrix is a vital determinant of mitochondrial dynamic distribution, energy generation and other metabolic functions in neurons, as well as of neuronal survival. These roles of neuronal mitochondrial [Ca⁺⁺] are in part coupled to their capacity to buffer functionally critical increases in cytoplasmic concentration of Ca⁺⁺. Five neuronal MPs, that are involved in Ca⁺⁺ homeostasis, had abnormal NDEV levels in PASC relative to healthy Ctls (FIG. 2). MCU and NCLX are the principal channels for moving Ca⁺⁺ in and out of the mitochondrial matrix, respectively, and the Na′/Ca′ exchanger LETM1 is one of many alternative channels to MCU.

Meaningful levels of two functionally critical SARS-CoV-2 proteins were detected in NDEVs and their NDEV levels differed significantly among the subgroups of participants (Table 2). The mean NDE levels of SARS-CoV-2 proteins, S1 and N, were significantly higher in ADEVs and NDEVs of all affected subgroups than background levels in controls. Further, mean ADEV and NDEV levels of N distinguished PASC w/NP from PASC w/o NP and from COVID-19 w/o PASC. Levels of mitochondrial proteins and viral proteins in plasma NDEVs may be valuable biomarkers for the diagnosis of PASC and for evaluating effects of new treatment modalities.

The detection of abnormal levels of one or more ADEV and/or NDEV mitochondrial proteins or viral proteins described herein that appear during acute COVID-19 may accurately predict the risk of developing PASC and its likely severity and/or duration.

These results showed that abnormal levels of SARS-CoV-2 proteins and mitochondrial proteins were detected in plasma neuron-derived extracellular vesicles and astrocyte-derived extracellular vesicles from biological samples from COVID-19 subjects. These results demonstrated that the methods of the present disclosure are useful for detecting SARS-CoV-2 proteins and mitochondrial proteins levels in vesicles in biological samples. These results further demonstrated that the methods of the present disclosure may be used to detect SARS-CoV-2 proteins and mitochondrial proteins associated with pathogenesis of long COVID-19. These results further showed that methods of the present disclosure are useful for prognosis, diagnosis, treating or monitoring treatment of SARS-CoV-2 proteins and mitochondrial protein level abnormalities associated with COVID-19. The results suggested that the methods of the present disclosure would be useful for treating long COVID-19.

### Example 2: Detection of SARS-CoV-2 Proteins and Mitochondrial Proteins in Neuron-Derived Extracellular Vesicles (NDEVs) and Astrocyte-Derived Extracellular Vesicles (ADEVs) in Biological Samples from COVID-19 Patients

SARS-CoV-2 proteins and mitochondrial proteins in plasma neuron-derived extracellular vesicles (NDEVs) were quantified in biological samples from COVID-19 patients as follows. Participants with nucleic acid assay-confirmed SARS-CoV-2 infection were enrolled in two San Francisco-based cohorts: the Long-term Impact of Infection with Novel Coronavirus (LIINC) COVID-19 long-term cohort (NCT04362150; www.liincstudy.org) and the FIND-COVID household-based acute COVID-19 cohort (findcovid19.org) 23, 24. Participant data and biospecimens were collected between April, 2020 and December, 2021 during the waves of COVID-19 caused by original strains but before predominance of the Omicron variants. Samples from weekly blood-draws are available during the acute phase and then monthly up to four months during the period of PASC for additional longitudinal studies. Four patients with acute COVID-19 from other ambulatory services of the University of California were enrolled and evaluated similarly, two who developed PASC and two who resolved their infection without PASC (Table 4). The initial evaluation of all included confirmation of prior or current SARS-CoV-2 RNA positivity, a range of relevant basic clinical laboratory studies, and detailed assessment of acute and post-acute COVID-19-attributable symptoms by a clinical research coordinator and/or infectious disease specialist physician.

Study instruments encompassed questions about current symptoms, preexisting co-morbidities and disabilities due to the present illness. At the initial and all subsequent visits, participants completed a general Patient Health Questionnaire (PHQ) and those with any NP issues also completed several specific questionnaires Relevant neuropsychiatric (NP) manifestations tabulated were irritability or agitation determined by the Patient Health Questionnaire (PHQ) and anxiety or depression delineated by the PHQ-8 and General Anxiety Disorder-7 (GAD-7) questionnaires using instruments that had been developed to track symptoms from the acute into the long-term phases. PASC symptoms were defined as symptoms of acute COVID-19 that continued beyond four weeks after onset and/or new symptoms attributable to COVID-19 that appeared beyond four weeks after onset. NP manifestations and abnormal NDEV levels of SARS-CoV-2 proteins and mitochondrial proteins, as well as a few selected plasma immune-inflammatory cytokines have been reported for some of the long-term patients (Table 3).

TABLE 3
Demographic and clinical characteristics of participant groups assessed in the post-acute
phase of COVID-19.

| | | | | | Severe |
|---|---|---|---|---|---|
| | | Acute COVID- | PASC w/o | PASC w/ | PASC w/ |
| | Control | 19 w/o PASC | NP | NP | NP |
| Number | 12 | 8 | 15 | 15 | 8 |
| Mean age (range) | 48 (26-67) | 42 (30-54) | 46 (34-56) | 34 (31-46) | 49 (44-52) |
| Female/male/TG male | 9/3/0 | 8/0/0 | 9/6/0 | 9/6/0 | 7/0/1 |
| Race, ethnicity | 4/3/1/4/0 | 2/5/0/1/0 | 5/7/0/2/1 | 3/10/1/0/1 | 3/5/0/0/0 |
| H/W/B/A/O | | | | | |
| Days from symptom | N/A | 57 (35-64) | 62 (35-81) | 60 (37-80) | 60 (44-84) |
| onset to blood | | | | | |
| sampling, mean (range) | | | | | |

H, Hispanic;
W, White;
B, Black;
A, Asian;
O, other;
TG, transgender;
NP, neuropsychiatric
manifestations;
w/, with;
w/o, without.

TABLE 4
Participants in the acute phase of COVID-19 who resolved infection without PASC (No
PASC) or developed PASC (PASC) and matched controls without COVID-19 infection.

| | | | | Total number of | |
|---|---|---|---|---|---|
| | Age | | | PASC | |
| | (years, | | Race, ethnicity | symptoms | |
| Study Group | mean ± SEM) | Females/Males | H/W/B/A/O | (range) | NP symptoms |
| Controls (n = | 39.3 ± 3.88 | 5/5 | 3/5/1/1/0 | 0 | 0 |
| 10) | | | | | |
| No PASC | 35.9 ± 2.93 | 6/4 | 4/4/1/1/0 | 0 | 0 |
| (n = 10) | | | | | |
| PASC (n = 10) | 35.3 ± 3.96 | 5/5 | 5/3/0/2/0 | 1-10 | 6/10 |

NP = neuropsychiatric symptoms; summary of results of PHQ8 and GAD7 tests.
H, Hispanic;
W, White;
B, Black;
A, Asian;
O, other.

Six groups of SARS-CoV-2-infected patients were assembled for the study, in addition to separate groups of controls (Ctls) who donated plasma before the current pandemic and were age- and sex-matched to infected patients: Table 3-a) at least four weeks since the onset of acute COVID-19 with no fever, overall improvement in symptoms for at least two weeks, and no evidence of PASC, b) PASC without (w/o) neuropsychiatric (NP) symptoms, c) PASC with (w/) NP symptoms (<7 symptoms), d) severe PASC w/NP symptoms (>7 symptoms); Table 4-a) acute COVID-19 within nine days of detection that later evolved into PASC and f) acute COVID-19 that resolved with no evidence of PASC.

Isolation of Neuron-Derived Extracellular Vesicles (NDEVs)

Portions of 0.25 ml of thawed EDTA-plasma from the PASC period of each group (Table 3) or in the first 9 days of acute COVID-19 (Table 4) were incubated with thromboplastin D (ThermoFisher Scientific, Waltham, Mass.) and treated with protease inhibitor cocktail (Roche, Indianapolis, Ind.) and phosphatase inhibitor cocktail (Thermo Fisher Scientific; DBS⁺⁺). After centrifugation at 3000×g for 30 minutes at 4° C., total extracellular vesicles (EVs) were harvested from resultant supernatants by precipitation with 126 μL per tube of ExoQuick (System Biosciences, Mountain View, Calif.) and centrifugation at 1500×g for 30 minutes at 4° C.

Isolation of Neuron-Derived Extracellular Vesicles (NDEVs)

Portions of 0.25 ml of thawed EDTA-plasma from the PASC period of each group (Table 3) or in the first 9 days of acute COVID-19 (Table 4) were incubated with thromboplastin D (ThermoFisher Scientific, Waltham, Mass.) and treated with protease inhibitor cocktail (Roche, Indianapolis, Ind.) and phosphatase inhibitor cocktail (Thermo Fisher Scientific; DBS⁺⁺). After centrifugation at 3000×g for 30 minutes at 4° C., total extracellular vesicles (EVs) were harvested from resultant supernatants by precipitation with 126 μL per tube of ExoQuick (System Biosciences, Mountain View, Calif.) and centrifugation at 1500×g for 30 minutes at 4° C.

To enrich NDEVs including neuron-derived exosomes (NDEs), replicate preparations of total plasma EVs were resuspended in 0.35 mL of DBS⁺⁺ with 2.0 μg of mouse anti-human CD171 (L1CAM neural adhesion protein) biotinylated monoclonal antibody (clone 5G3; eBiosciences, San Diego, Calif.) or 2.0 μg of mouse anti-human NCAM1 biotinylated monoclonal antibody (Santa Cruz Biotechnologies sc-51742) in 50 μL of 3% bovine serum albumin (BSA; 1:3.33 dilution of Blocker BSA 10% solution in DBS; ThermoFisher Scientific) per tube. After this incubation, 10 μL of streptavidin-agarose Ultralink resin (ThermoFisher Scientific) in 40 μL of 3% BSA were added to each tube followed by a second incubation as described (Goetzl EJ, et al. Neurology 85, 40-47 (2015) and Goetzl EJ, et al. Faseb J30, 4141-4148 (2016)). After centrifugation at 800×g for 10 minutes at 4° C. and removal of the supernatant, each pellet with NDEVs was suspended in 100 μL of cold 0.05 M glycine-HCl (pH 3.0) for 5 minutes and centrifuged at 4000×g for 10 minutes, all at 4° C. Glycine-HCl supernatants then were transferred to clean tubes containing 25 μL of 10% BSA and 10 μL of 1 M Tris-HCl (pH 8.0) and mixed gently. An aliquot of 5 μL was removed from each tube for NEV counts before addition of 370 μL of mammalian protein extraction reagent (M-PER, ThermoFisher Scientific).

For counting and sizing of extracellular vesicles, each suspension was diluted 1:50 in PBS. The mean diameter (nanometers) and concentration (particles per milliliter) of EVs in each suspension were determined by nanoparticle tracking analysis (NTA) using the Nanosight NS500 system with a G532 nm laser module and NTA 3.1 nanoparticle tracking software (Malvern Instruments, Malvern, United Kingdom) as described in Mustapic M, et al. Front Neurosci 11, 278 (2017).

Permeabilization of NDEVs, Antibody Labeling of their Proteins and Bioimage Analyses

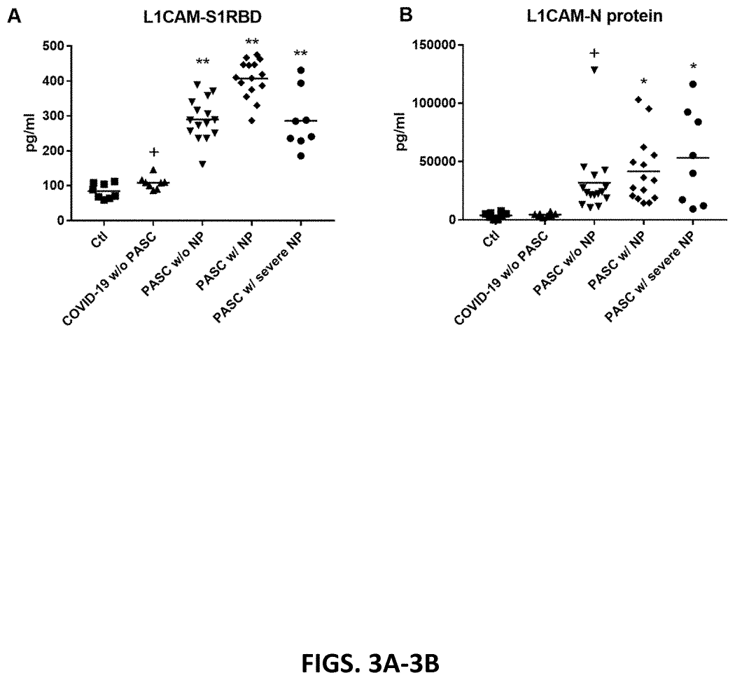

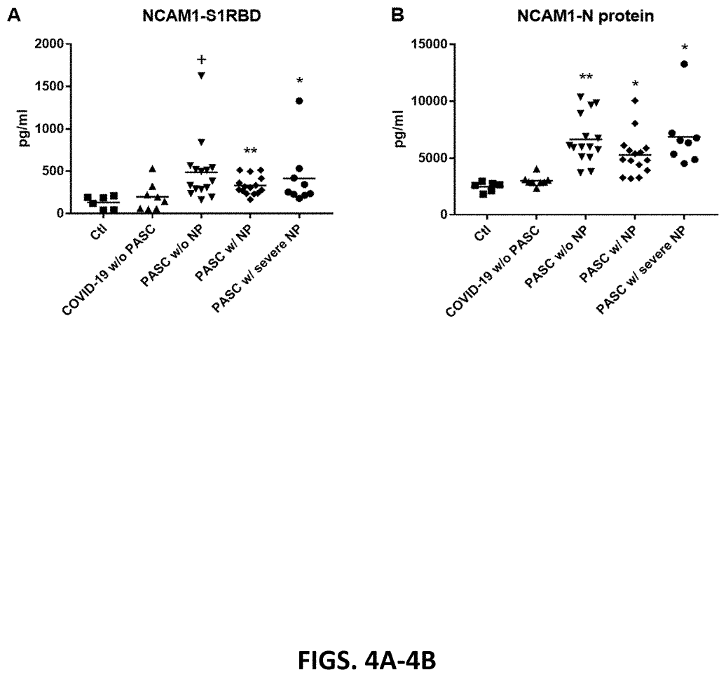

NDEVs first were anchored to plate wells by anti-L1CAM antibody (FIG. 3) or anti-NCAM-1 antibody (FIG. 4), permeabilized with 0.01% Triton X-100, and then incubated serially with rabbit biotin-labeled anti-S1 RBD protein antibody (FIG. 3A, 4A) or rabbit biotin-labeled anti-N protein antibody (FIG. 3B, 4B), streptavidin-horse radish peroxidase conjugate and tetramethylbenzidine (TMB) substrate (RayBiotech Life, Peachtree Corners, GA). After adding Stop Solution, the optical density of each plate well was determined at 450 nm and these values were converted to pg/ml with standard curves generated concurrently with one strip of plate wells each from ELISAs for S1 RBD protein and N protein (RayBiotech Life).

For imaging studies, human plasma NDEVs were isolated as described in Mustapic M, et al. Front Neurosci 11, 278 (2017), permeabilized in 0.001% Triton X-100 in PBS for 5 minutes at room temperature, supplemented with polyethylene glycol 10000 (Sigma, 92897) at a final concentration of 10% and precipitated by centrifugation at 3500×g for 10 minutes at room temperature (Mondal A, et al. Biol Proced Online 21, 4 (2019)). The pellet of permeabilized NDEVs was re-suspended in multiple aliquots of 200 uL of PBS for labeling with one or more of the following primary antibodies: rabbit anti-L1CAM monoclonal antibody (Abcam ab208155), mouse anti-NCAM1 monoclonal antibody (Santa Cruz Biotechnologies sc-51742), mouse anti-CoV-1/2 spike protein monoclonal antibody (Cell Signaling Technology 52342) or rabbit anti-CoV-2 S1 RBD antibody (RayBiotech 130-10759B), mouse anti-CoV-1/2 nucleocapsid [N] protein monoclonal antibody (Cell Signaling Technology 68344) or rabbit anti-CoV-2 N protein antibody (RayBiotech 130-10760B); all antibody mixtures were incubated overnight at 4° C. with rocking. The mixtures then were centrifuged at 3500×g for 10 minutes at room temperature and the pellets washed with 200 uL of 10% polyethylene glycol 10000 for three cycles.

Alexa Fluor 488- or 568-conjugated second antibodies (ThermoFisher Scientific) were added to the primary antibody-labeled NDEV suspensions at 1:200 (v:v) followed by incubation for 60 minutes in the dark at room temperature. After three cycles of polyethylene glycol 10000 precipitation and washing as for removing unbound primary antibodies, labeled NDEV suspensions were incubated with Capto Core 400 resin (1:1, v:v) for 30 minutes. The labeled NDEVs were precipitated in 10% polyethylene glycol 10000 and resuspended in 20 uL of PBS. Then 10 uL of each suspension was placed on a glass slide to which was added a drop of Prolong Anti-Fade Mountant (ThermoFisher Scientific) before sealing with a glass coverslip and imaging within 24 hours.

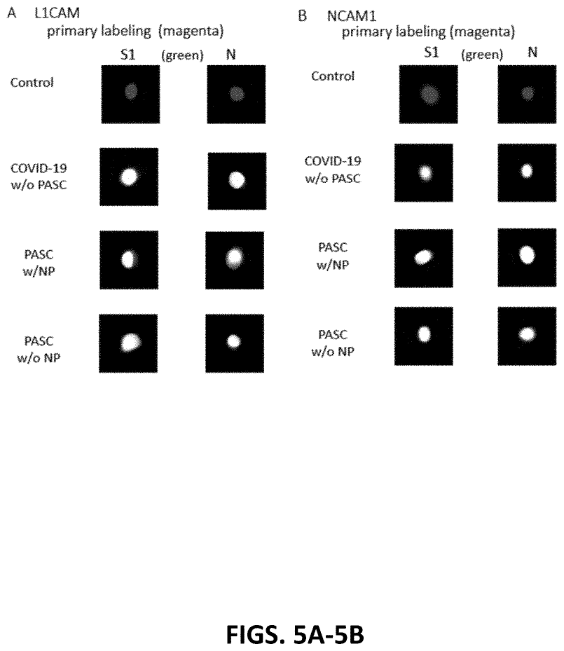

Immunofluorescence images of labeled NDEVs were acquired with a 100x/1.40 numerical aperture oil objective lens on a Zeiss LSM 980 confocal microscope with Airyscan. Lasers were used at 2% or less of power; 488 nm laser for Alexa Fluor 488 and 561 nm laser for Alexa Fluor 568. Detection gain was adjusted to cover the full dynamic range and avoid saturated pixels. All images were acquired at a 1,024×1,024 pixel xy resolution (FIG. 5).

Quantification of NDEV Proteins

Human NDEV proteins were quantified by enzyme-linked immunosorbent assay (ELISA) kits for tetraspannin exosome marker CD81, 16S rRNA-encoded Humanin (CUSABIO by American Research Products, Waltham, Mass.), N-methyl-D-aspartate receptor 1 (NMDAR1) (Novus Biologicals, LLC, Centennial, CO), subunit 10 of cytochrome b-cl oxidase (respiratory chain complex III) (CIII-10) (Abbkine Scientific Co., Ltd. by American Research Products), mitochondrial open-reading frame of the 12S rRNA-c (MOTS-c) (Cloud-Clone Corp. by American Research Products), SARS-CoV-2 protein S1 (RBD), SARS-CoV-2 protein N (Abcam, Inc., Waltham, Mass. and Raybiotech Life, Inc., Peachtree Corners, GA), syntaphilin (SNPH), Sterile Alpha and TIR motif-containing protein 1 (SARM-1), voltage-dependent anion-selective channel protein 1 (VDAC1) (Wuhan FineTest Biotech Co. by American Research Products and Abbexa, Ltd., Cambridge, UK), and transcription factor A mitochondrial (TFAM) (Aviva Systems Biology, San Diego, Calif.). The mean value for all determinations of CD81 in each assay group was set at 1.00, and relative values of CD81 for each sample were used to normalize their recovery. All ELISAs were performed by one investigator (EJG) without knowledge of group identity of any subject.

Statistics

After establishing that data were normally distributed, the significance of differences between COVID-19 patient and control levels as well as between patient group levels were calculated by an unpaired t test (Graphpad Holdings, LLC, San Diego, Calif.). Significance based on p values was assessed after Bonferroni correction. Pearson Correlation Coefficient analysis was performed to assess relationships between NDEV counts and NDEV levels of CD81 (Graphpad Prism 9). There were no adjustments for potential confounding variables.

The participants in all sub-groups were predominantly middle aged and of Hispanic ethnicity and Caucasian race (Tables 3 and 4). Participants studied four weeks or longer after acute COVID-19 consisted of four groups (Table 3): no PASC (n=8), PASC (1-7 symptoms) w/o NP (n=15), PASC (1-7 symptoms) w/NP (n=15), and severe PASC (>7 symptoms) w/NP (n=8). Participants with acute COVID-19 consisted of two groups (Table 4): PASC did not develop subsequently termed no PASC (n=10) and PASC did develop later termed PASC (n=10). None of the participants had received any SARS-CoV-2 vaccine by the time of enrollment and blood donation. Pulmonary involvement was similarly prevalent in each group with acute COVID-19 (Table 4). All participants with acute COVID-19 who developed PASC, but none who recovered without PASC, had NP abnormalities (Table 4).

Properties of NDEVs Analyzed in Acute and Post-Acute COVID-19

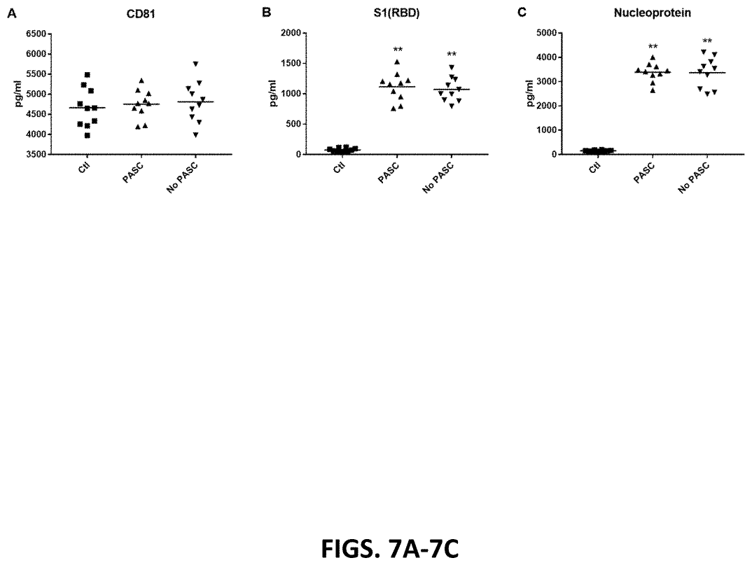

Counts of NDEVs at the time of acute COVID-19 (Table 4) in preparations from those who would develop PASC (11.5±1.17×10¹⁰/ml) and those who would not develop PASC (10.8±0.96×10¹⁰/ml) were statistically no different from each other or different from the control group (10.9±0.88×10¹⁰/ml) and paralleled the similar levels of the exosome marker CD81 for each group (FIG. 7A). For NDEVs obtained in the post-acute phase, counts of preparations from the PASC w/o NP sub-group and the PASC with severe NP sub-group were respectively significantly higher and lower than in controls (Table 3) and also paralleled the levels for the exosome marker CD81 (Peluso MJ, et al. Ann Neurol 91, 772-781 (2022)). Counts of NDEVs in preparations from the PASC w/o NP sub-group were significantly higher than those of the PASC w/NP subgroup, as were the levels of CD81 (Table 3) (Peluso MJ, et al. Ann Neurol 91, 772-781 (2022)). The Pearson Correlation Coefficient for the comparison of NDEV counts with CD81 levels for participants of all groups was 0.9614 (p<0.0001). CD81 levels of all subgroups in the PASC (Table 3) and acute COVID-19 (Table 4) clusters thus were used to normalize levels of analytes as has been described in Mustapic M, et al. Front Neurosci 11, 278 (2017).

Levels of SARS-CoV-2 S1 (RBD) and N Proteins in Permeabilized NDEVs During PASC

Plasma NDEVs from all three groups of post-acute participants with PASC (Table 3) isolated by absorption to plate wells coated with anti-L1CAM antibody and permeabilized showed significantly higher levels of S1 RBD protein and N protein than those from COVID-19-negative controls (FIG. 3A, 3B). In contrast, NDEVs isolated by anti-L1CAM antibody absorption from plasmas of patients who earlier had COVID-19, but no manifestations of PASC in the same later time period as the three groups of PASC patients, had marginally higher levels of S1 RBD protein but not N protein relative to controls, which suggests almost complete clearance of SARS-CoV-2 from neuronal sources. To demonstrate that these findings were not limited to L1CAM-positive NDEVs, similar assays were performed with NDEVs bearing another neuronal marker NCAM-1. Plasma NDEVs isolated from the same three groups of PASC patients by absorption to plate wells coated with anti-NCAM-1 antibody similarly showed significantly higher levels of S1 RBD protein and N protein than those from COVID-19-negative controls (FIG. 4A, 4B). NDEVs isolated by anti-NCAM-1 antibody absorption from plasmas of patients who had COVID-19, but no manifestations of PASC in the same later time period as the three groups of PASC patients, had levels of S1 RBD protein and N protein levels no higher than controls. These findings also suggested complete clearance of SARS-CoV-2 from their neuronal sources.

In a separate approach to image SARS-CoV-2 proteins in plasma NDEVs, suspensions of total plasma EVs were permeabilized by Triton x-100 and identified by staining with anti-L1CAM antibodies (FIG. 5A) or anti-NCAM1 antibodies (FIG. 5B) prior to a second labeling with antibodies to S1 (RBD) and N proteins. The representative examples shown demonstrate qualitatively that those from both groups of patients with PASC w/ and w/o NP (Table 3) and those from patients at the same late stage following acute COVID-19, but without any manifestations of PASC, all have detectable but technically unquantifiable amounts of intra-NDEV S1 and N proteins. In contrast, both representative NDEVs identified by antibodies for L1CAM and NCAM1 in plasma of control subjects with no history or evidence of COVID-19 contain neither S1 or N proteins (FIG. 5A, 5B).

Levels of SARS-CoV-2 S1 (RBD) and N Proteins in NDEV Extracts in Acute COVID-19

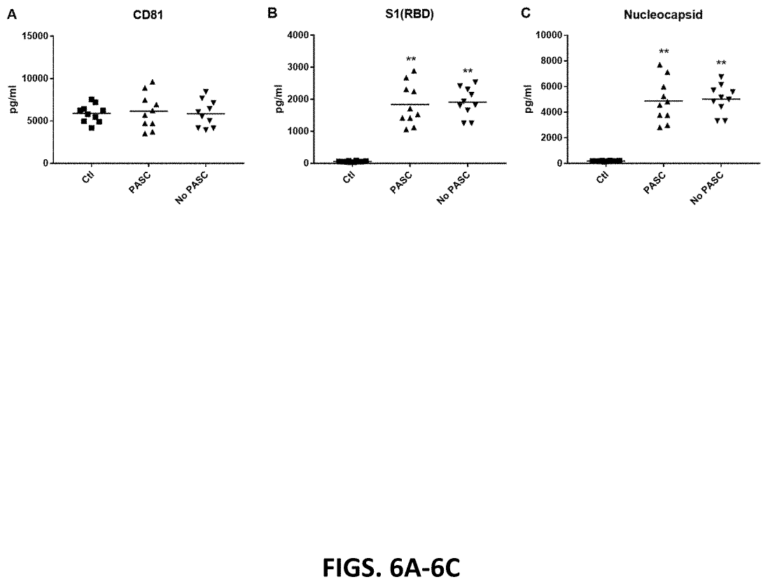

After identifying SARS-CoV-2 proteins in NDEVs of the post-acute COVID-19 cohort by several means, we chose the most consistently reliable to quantify these potentially predictive biomarkers of PASC in NDEV extracts from acutely infected patients at 9 days after the onset of COVID-19 infections and uninfected controls (Table 4), NDEVs were isolated with anti-L1CAM antibodies (FIG. 6) or anti-NCAM1 antibodies (FIG. 7) and their proteins extracted for ELISAs. Extracts of NDEVs from control and acutely infected subjects who did or did not develop PASC had the same levels of CD81 whether isolated with anti-L1CAM antibodies (FIG. 6A) or anti-NCAM1 antibodies (FIG. 7A). Levels of S1 (RBD) (FIGS. 6B and 7B) and Nucleocapsid (N) (FIGS. 6C and 7C) proteins were similarly significantly higher in extracts of NDEVs isolated with either antibody from acutely infected subjects of both PASC and no PASC sets than those of uninfected controls. In the group who developed PASC, the NDEV levels of S1 (RBD) in acute COVID-19 were elevated to similar levels as they were later with established PASC, but at this later time in the no PASC group the NDEV levels of S1 (RBD) were in the control range (Peluso MJ, et al. Ann Neural 91, 772-781 (2022)). In contrast, the NDEV levels of N for the group that later developed PASC were five- to six-fold higher in the time period of established PASC than in acute COVID-19, but were significantly lower at this later time than in acute COVID-19 for the no PASC group (Peluso MJ, et al. Ann Neural 91, 772-781 (2022)). There were no differences between acute COVID-19 NDEV levels of either SARS-CoV-2 protein for groups that would (PASC) or would not (No PASC) later develop PASC (Table 4).

Levels of Mitochondrial Proteins in NDEV Extracts in Acute COVID-19

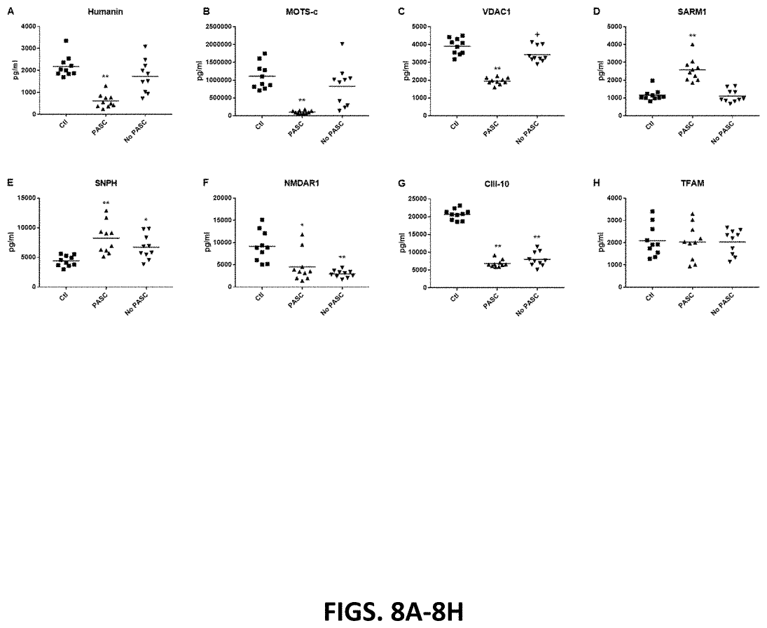

As acutely-infected patients who would or would not subsequently develop PASC had similarly elevated levels of SARS-CoV-2 proteins in NDEVs (FIGS. 6, 7), NDEV levels of a range of mitochondrial proteins that have been found to reflect host responses to COVID-19 were assessed in the same groups of participants (Peluso MJ, et al. Ann Neural 91, 772-781 (2022)). Of the eight mitochondrial proteins quantified in NDEV extracts, levels of seven were significantly different at the time of acute COVID-19 in those who would later develop PASC than in uninfected controls, as they had been when measured later in established PASC (FIG. 8) (Peluso MJ, et al. Ann Neural 91, 772-781 (2022)). NDEV extract levels of the neuronal protective and metabolic regulatory peptides humanin and MOTS-c, that are produced in mitochondria, the outer mitochondrial membrane ion channel VDAC1, and the mitochondrial NADase with axonal degenerative activity SARM-1 were significantly different during acute COVID-19 destined to develop into PASC than in uninfected controls and were significantly different (p<0.001 for all four proteins) in those destined to develop PASC than in those whose acute COVID-19 would resolve without progressing to PASC (No PASC, FIG. 8). In contrast, NDEV extract levels of NMDAR1, SNPH and CIII-10 were significantly different during acute COVID-19 in both the groups that would develop PASC and those who would recover without PASC relative to those of uninfected controls. However, none of the differences between levels of these proteins in subjects destined to develop PASC and those whose acute COVID-19 would resolve without progressing to PASC was significant. NDEV extract levels of TFAM were unaffected by COVID-19.

These results showed that abnormal levels of SARS-CoV-2 proteins and mitochondrial proteins were detected in plasma neuron-derived extracellular vesicles from biological samples from COVID-19 subjects. These results demonstrated that the methods of the present disclosure are useful for detecting SARS-CoV-2 proteins and mitochondrial proteins levels in vesicles in biological samples. These results further demonstrated that the methods of the present disclosure may be used to detect SARS-CoV-2 proteins and mitochondrial proteins associated with pathogenesis of long COVID-19. These results further showed that methods of the present disclosure are useful for prognosis, diagnosis, treating or monitoring treatment of SARS-CoV-2 proteins and mitochondrial protein level abnormalities associated with COVID-19. The results suggested that the methods of the present disclosure would be useful for treating long COVID-19.

Various modifications of the disclosure, in addition to those shown and described herein, will become apparent to those skilled in the art from the foregoing description. Such modifications are intended to fall within the scope of the appended claims.

All references cited herein are hereby incorporated by reference herein in their entirety.

What is claimed is:

1. A method comprising: a) providing a biological sample comprising vesicles from a subject; b) enriching the sample for vesicles; and c) detecting the presence of one or more biomarkers, wherein the one or more biomarker is a SARS-CoV-2 protein and/or mitochondrial protein.

2. The method of claim 1, wherein the one or more biomarker is CD81, syntaphilin (SNPH), subunit 6 of NADH-ubiquinone oxidoreductase (respiratory chain complex I) (CI-6), subunit 10 of cytochrome b-cl oxidase (respiratory chain complex III) (CIII-10), Humanin, myosin VI (MYO6), mitochondrial open-reading frame of the 12S rRNA-c (MOTS-c), voltage-dependent anion-selective channel protein 1 (VDAC1), N-methyl-D-aspartate receptor 1 (NMDAR1), mitochondrial calcium uniporter (MCU), solute carrier family 8 member B1 (SLC24A6) or sodium/calcium exchanger (NCLX), translocator protein (TSOP), leucine zipper EF-hand containing transmembrane 1 protein (LETM1), SARS-CoV-2 S1 (RBD), and/or SARS-CoV-2 nucleocapsid (N).

3. The method of claim 1, wherein the vesicles are neuron-derived extracellular vesicles (NDEVs) and/or astrocyte-derived extracellular vesicles (ADEVs).

4. The methods of claim 1, wherein the biological sample is selected from the list consisting of whole blood, plasma, serum, lymph, amniotic fluid, urine, and saliva.

5. The method of claim 1, wherein the subject has or is suspected of having COVID-19.

6. The method of claim 1, wherein the subject has or is suspected of having or developing long COVID-19.

7. A method comprising: a) providing a biological sample comprising vesicles from a subject currently or previously infected with COVID-19; b) isolating vesicles from the biological sample; and c) detecting the presence of one or more biomarkers in the vesicles, wherein the one or more biomarker is a SARS-CoV-2 protein and/or mitochondrial protein.

8. The method of claim 7, wherein the isolating vesicles from the biological sample comprises: contacting the biological sample with an agent under conditions wherein the vesicles present in the biological sample bind to the agent to form an vesicle-agent complex; and isolating the vesicles from the vesicle-agent complex to obtain a sample containing the vesicles, wherein the purity of the vesicles present in said sample are greater than the purity of the vesicles present in said biological sample.

9. The method of claim 7, wherein the vesicles are neuron-derived extracellular vesicles (NDEVs) and/or astrocyte-derived extracellular vesicles (ADEVs).

10. The method of claim 7, wherein the one or more biomarker CD81, syntaphilin (SNPH), subunit 6 of NADH-ubiquinone oxidoreductase (respiratory chain complex I) (CI-6), subunit 10 of cytochrome b-cl oxidase (respiratory chain complex III) (CIII-10), Humanin, myosin VI (MYO6), mitochondrial open-reading frame of the 12S rRNA-c (MOTS-c), voltage-dependent anion-selective channel protein 1 (VDAC1), N-methyl-D-aspartate receptor 1 (NMDAR1), mitochondrial calcium uniporter (MCU), solute carrier family 8 member B1 (SLC24A6) or sodium/calcium exchanger (NCLX), translocator protein (TSOP), leucine zipper EF-hand containing transmembrane 1 protein (LETM1), SARS-CoV-2 S1 (RBD), and/or SARS-CoV-2 nucleocapsid (N).

11. The method of claim 8, wherein the agent is an antibody.

12. The method of claim 11, wherein the antibody is an anti-human CD171 (L1CAM neural adhesion protein) antibody or an anti-human glutamine aspartate transporter (GLAST) (ACSA-1) antibody.

13. The methods of claim 7, wherein the biological sample is selected from the list consisting of whole blood, plasma, serum, lymph, amniotic fluid, urine, and saliva.

14. The method of claim 7, wherein the detecting the presence of the marker in the biological sample comprises detecting the amount of the marker in the biological sample.

15. The method of claim 7, further comprising the step of determining a treatment course of action based on the detection of the one or more biomarkers.

16. The method of claim 7, wherein the subject has or is suspected of having COVID-19.

17. The method of claim 7, wherein the subject has or is suspected of having or developing long COVID-19.

18. A method for treating a subject for long COVID-19, comprising the steps of: providing a biological sample from a subject currently or previously infected with COVID-19, wherein the sample comprises vesicles; measuring the level of one or more biomarkers selected from the group consisting of SARS-CoV-2 protein and/or mitochondrial protein from the biological sample, wherein an altered level of the one or more biomarkers in the sample relative to the level in a control sample is indicative of a need for treatment; and administering an effective amount of an agent to the subject thereby treating the long COVID-19 in the subject.

19. The method of claim 18, wherein the one or more marker is CD81, syntaphilin (SNPH), subunit 6 of NADH-ubiquinone oxidoreductase (respiratory chain complex I) (CI-6), subunit 10 of cytochrome b-cl oxidase (respiratory chain complex III) (CIII-10), Humanin, myosin VI (MYO6), mitochondrial open-reading frame of the 12S rRNA-c (MOTS-c), voltage-dependent anion-selective channel protein 1 (VDAC1), N-methyl-D-aspartate receptor 1 (NMDAR1), mitochondrial calcium uniporter (MCU), solute carrier family 8 member B1 (SLC24A6) or sodium/calcium exchanger (NCLX), translocator protein (TSOP), leucine zipper EF-hand containing transmembrane 1 protein (LETM1), SARS-CoV-2 S1 (RBD), and/or SARS-CoV-2 nucleocapsid (N).

20. The method of claim 18, wherein the vesicles are neuron-derived extracellular vesicles (NDEVs) and/or astrocyte-derived extracellular vesicles (ADEVs).
